(19) United States
(12) Patent Application Publication
(10) Pub. No.: US 2021/0391083 A1
(43) Pub. Date: Dec. 16, 2021

(54) METHOD FOR PROVIDING HEALTH THERAPEUTIC INTERVENTIONS TO A USER

(71) Applicant: Ginger.io, Inc., San Francisco, CA (US)

(72) Inventors: Sai Moturu, San Francisco, CA (US); Anmol Madan, San Francisco, CA (US); Greg Elliott, San Francisco, CA (US); Amanda Withrow, San Francisco, CA (US); Shishir Dash, San Francisco, CA (US)

(21) Appl. No.: 17/463,432

(22) Filed: Aug. 31, 2021

Related U.S. Application Data
(63) Continuation-in-part of application No. 15/265,454, filed on Sep. 14, 2016, now abandoned.
Application No. 15/265,454 is a continuation-in-part of application No. 13/969,339, filed on Aug. 16, 2013, now abandoned.
(60) Provisional application No. 61/683,867, filed on Aug. 16, 2012.
(60) Provisional application No. 61/683,869, filed on Aug. 16, 2012.
(60) Provisional application No. 62/218,848, filed on Sep. 15, 2015.

(51) Int. Cl. G16H 50/20; G16H 10/20; G16H 40/67; H04L 12/58; G09B 19/00

(52) U.S. Cl. CPC G16H 50/20; G16H 10/20; G16H 50/30; H04L 51/04; G09B 19/00; G16H 40/67

(57) ABSTRACT

A method and system for digitally providing healthcare to a patient, the method including receiving a log of use dataset associated with patient digital communication behavior at a mobile computing device, wherein the first log of use dataset corresponds to a time period; receiving a supplementary dataset corresponding to the time period; receiving a survey response dataset from the patient, the survey response dataset corresponding to the time period; receiving a care provider dataset in association with the time period; selecting a therapeutic intervention from a set of therapeutic interventions, based on processing with at least one of the first log of use dataset, the supplementary dataset, the survey response dataset, and the care provider dataset; generating a dynamic care plan modifiable over a time period; promoting the therapeutic intervention according to the dynamic care plan.

### CROSS-REFERENCE TO RELATED APPLICATIONS

This application is a continuation-in-part of U.S. application Ser. No. 15/265,454, filed 14 Sep. 2016, which claims the benefit of U.S. Provisional Application No. 62/218,848 filed 15 Sep. 2015, and which is a continuation-in-part of U.S. application Ser. No. 13/969,339 filed 16 Aug. 2013, which claims the benefit of U.S. Provisional Application Ser. No. 61/683,867 filed on 16 Aug. 2012 and U.S. Provisional Application Ser. No. 61/683,869 filed on 16 Aug. 2012, which are each incorporated in its entirety herein by this reference.

### TECHNICAL FIELD

This invention relates generally to the field of healthcare and more specifically to a new and useful method for providing health therapeutic interventions to a user in the healthcare field.

### BACKGROUND

Life event triggers and other factors contributing to adverse psychological and/or physiological states can result in a combination of symptoms that interfere with a person's ability to work, sleep, study, eat, and enjoy once-pleasurable activities. For some individuals diagnosed with a disorder or a condition, access to therapy is limited, and the processes of receiving appropriate forms of therapy are often fraught with unnecessary inefficiencies. Timely/early therapeutic intervention in many forms of disease progression is crucial to affecting patient/user outcomes; however, timely therapeutic intervention requires intensive patient assessment and monitoring. Current systems and methods for monitoring patients or users exhibiting symptoms of conditions that affect psychological and/or physical states have some ability to influence patient outcomes, but are typically time intensive, cost-intensive, and/or entirely fail to identify when a patient/user is entering a critical state of a condition at which therapeutic intervention would be most effective. Furthermore, therapeutic interventions provided in relation to a patient/user state are often provided with sub-optimal timing, with therapeutic intervention type provided in a manner that is not dynamic in relation to idiosyncrasies of the user, preferences of the user, schedules of the user, and other factors. As such, current standards of detection, diagnosis and treatment of many disorders and conditions, as well as barriers (e.g., social barriers) to seeking diagnosis and treatment, are responsible for delays in diagnoses of disorders and/or misdiagnoses of disorders, which cause such disorders and conditions to remain untreated. Furthermore, such standards result in a reactionary approach, as opposed to a preventative approach to a critical event. In addition to these deficiencies, further limitations in detection, diagnosis, treatment, and/or monitoring of patient progress during treatment prevent adequate care of patients with diagnosable and treatable conditions.

As such, there is a need in the field of healthcare for a new and useful method and system for providing health therapeutic interventions to a user in the healthcare field. This invention creates such a new and useful and system.

### BRIEF DESCRIPTION OF THE FIGURES

FIGS. 1A-1B are flowcharts of variations of an embodiment of a method for digitally providing healthcare to a patient;

FIGS. 2A-2B are schematic representations of a method for digitally providing healthcare to a patient;

FIGS. 3A-3B depict schematic representations of variations of a method for digitally providing healthcare to a patient;

FIG. 4 depicts an example of a health tip therapeutic intervention in an embodiment of a method for digitally providing healthcare to a patient;

FIGS. 5A-5G depict examples of mindfulness activities in an embodiment of a method for digitally providing healthcare to a patient;

FIG. 6A-6G depict examples of a relaxation kit therapeutic intervention in an embodiment of a method for digitally providing healthcare to a patient;

FIG. 7 depicts an example of a body-awareness therapeutic intervention in an embodiment of a method for digitally providing healthcare to a patient;

FIGS. 8A-8E depict examples of a sleep therapeutic intervention in an embodiment of a method for digitally providing healthcare to a patient;

FIG. 9 depicts an example of an therapeutic intervention regimen in an embodiment of a method for digitally providing healthcare to a patient;

FIG. 10 depicts an example of a portion of a method for digitally providing healthcare to a patient; and

FIGS. 11A-11B depicts schematic representations of digital surveys;

FIGS. 12A-12B depicts schematic representations of therapeutic interventions;

FIGS. 13-14 depict examples of portions of a method for digitally providing healthcare to a patient;

FIGS. 15-16 depict examples of therapeutic interventions;

FIG. 17 depicts a schematic representations of a variation of a method for digitally providing healthcare to a patient;

FIGS. 18A-18C depict an example of a continuously updated dynamic care plan; and

FIG. 19 depicts an embodiment of a system for digitally providing healthcare to a patient.

### DESCRIPTION OF THE EMBODIMENTS

The following description of the embodiments of the invention is not intended to limit the invention to these embodiments, but rather to enable any person skilled in the art to make and use this invention.

### 1. Overview

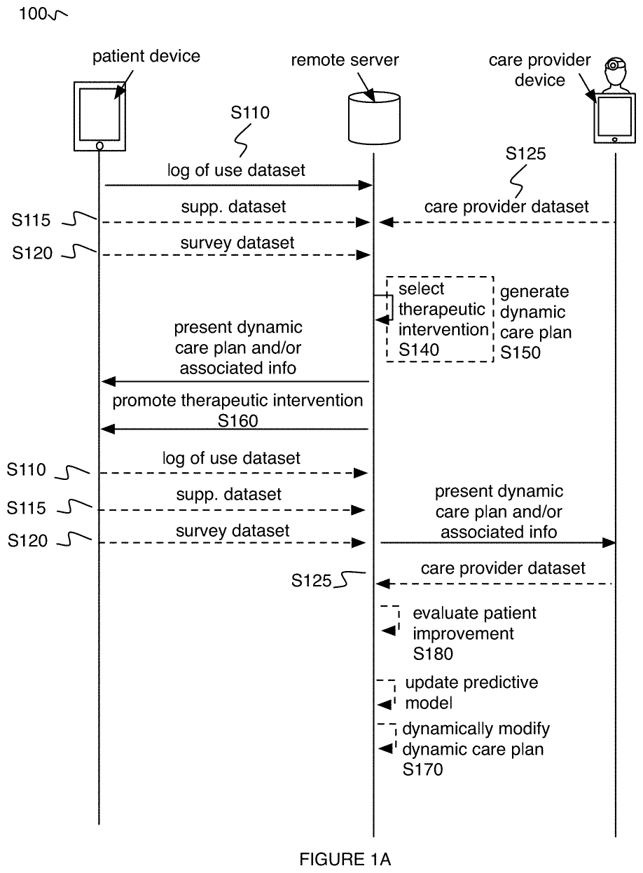

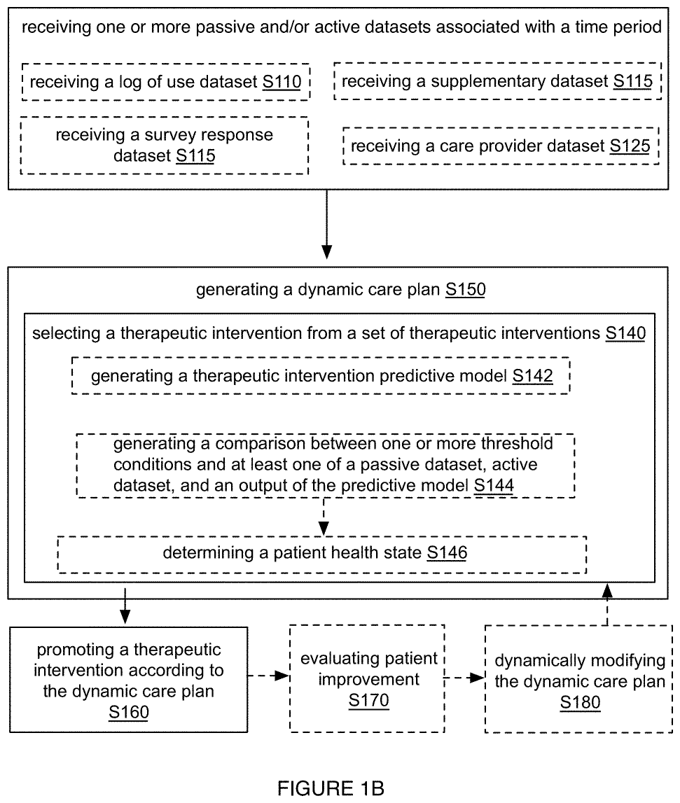

As shown in FIGS. 1A-1B, a method 100 for digitally providing healthcare to a patient includes: receiving a log of use dataset associated with patient digital communication behavior at a mobile computing device, wherein the first log of use dataset corresponds to a time period S110; receiving a supplementary dataset corresponding to the time period S115; receiving a survey response dataset from the patient, the survey response dataset corresponding to the time period S120; receiving a care provider dataset in association with the time period S125; selecting a therapeutic intervention from a set of therapeutic interventions, based on processing with at least one of the first log of use dataset, the supplementary dataset, the survey response dataset, and the care provider dataset S140; generating a dynamic care plan modifiable over a time period S150; promoting the therapeutic intervention according to the dynamic care plan S160. The method 100 can additionally or alternatively include dynamically modifying the dynamic care plan S170; and/or evaluating patient improvement S180.

The method 100 functions to detect a state of a user that could be improved with a therapeutic intervention. The method 100 additionally functions to provide the therapeutic intervention to the user at a time when the therapeutic intervention would be effective in improving the state of the user, and when the therapeutic intervention could be received or responded to by the user. The method 100 preferably detects the state of the user in a manner that improves therapeutic intervention provision effectiveness (e.g., with regard to a patient being symptomatic, with regard to detection prior to a patient entering a critical state, etc.). As such, implementation of the method 100 can result in improved patient outcomes through an improved assessment of patient states of need.

In generating a dynamic care plan for a patient, the method 100 preferably processes active data (e.g., survey responses, care provider data based on audio and/or textual communication with a patient, etc.), communication behavior (e.g., text messaging behavior, phone calling behavior, etc.), mobility behavior, and any other suitable supplementary information in order to determine the appropriate time(s) to provide therapeutic interventions pertaining to one or more patients, as well as the format(s) of provided therapeutic interventions. In variations, the method 100 can facilitate monitoring of states of a disorder or condition (e.g., a psychological disorder, a condition of depression, a pain-related condition, a sleep-related condition, a cardiovascular disease related condition, etc.), by enabling detection of changes in the patient's condition. In a specific application, the method 100 can monitor and analyze communication behavior, mobility behavior, and/or other behavior detected from any other suitable sensor(s) associated with a population of users over time, and provide therapeutic interventions to users for whom therapy is less accessible (e.g., due to cost, due to location, due to social barriers, etc.), in an effective, time-sensitive, and personalized manner.

Portions of the method 100 are preferably implemented in association with a non-generalized mobile computing device (e.g., a smartphone including mobility-related sensors such as an accelerometer, gyroscope, GPS module, light sensor, etc.) with digital communication capabilities (e.g., text messaging functionality, social media interactivity, phone calling capabilities, etc.). The method 100 can additionally or alternatively be implemented by a processing system in communication with a mobile computing devices associated with a set of users, for administering therapeutic interventions (e.g., health therapeutic interventions, clinical interventions) to the users in times of need.

### 2. Benefits

In specific examples, the method 100 and/or system 200 can confer several benefits over conventional methodologies used for generating a care plan that includes a therapeutic intervention. In specific examples, the method 100 and/or system 200 can perform one or more of the following:

First, the technology can provide an unobtrusive mechanism for multiple users/patients to receive appropriate therapeutic interventions (e.g., therapeutic interventions delivered through an application executing on a mobile computing device, health advice electronically delivered to a user, elements of a therapy program electronically delivered to a user, etc.), in a manner that accounts for indication frequency (e.g., per user, for a population of patients), precision in timing of provided therapeutic interventions, and therapeutic intervention content. As such, a personalized and user-modifiable care plan can be developed for the patient that delivers the appropriate interventions at the appropriate times while allowing improvement goals to be manually and/or automatically set. In particular, the therapeutic intervention(s) delivered to a user can further be based upon contextual information (e.g., a location of the patient, an assessment of “free time of the patient” based upon the user location/schedule/mobile device usage, etc.) pertaining to the user. In a specific example, the therapeutic interventions can educate a patient regarding his/her condition and how to appropriately manage it (e.g., through skill-building exercises for developing resilience to psychological symptoms). However, the method 100 can additionally or alternatively be implemented for any other suitable application.

Second, information derived from a population of users (i.e., patients) can be used to provide additional insight into connections between an individual user's behavior and risk of entering an adverse state (e.g., a critical episode of a condition), due to aggregation of data from the population of users. The aggregate data can then be used to improve therapeutic intervention predictive models for providing therapeutic interventions and/or to build improved features into an application executing the method 100. In examples, the population of users can include individuals characterized or grouped by any suitable demographics, any type of condition for which users can need help, any type of behavior in interacting with the system(s) implementing the method 100, and/or any other suitable feature.

Third, the technology can automatically provide personalized therapeutic interventions (e.g., personalized health tips, exercises, care provider matching, control of other patient devices, etc.) in the form of a dynamic care plan tailored for improving the health state of a patient. Patient responses to care plan can be monitored, evaluated, and used to dynamically modify a dynamic care plan for the patient. In some cases, for instance, care plans are dynamically adapted and evolved for each participant based on his or her actions and/or feedback in response to the dynamic care plan. As such, the technology can provide a full-stack approach to digitally monitoring the physiological and psychological health of a patient, leading to improved efficiency of care delivery, cost savings, and care delivery scalability.

Fourth, the technology can improve the technical fields of at least digital communication, computational modeling of user behavior, and personalized medicine. The technology can continuously collect and utilize datasets unique to internet-enabled, non-generalized mobile computing devices in order to provide personalized therapeutic interventions in real-time. Further, the technology can take advantage of such patient digital communication datasets to better improve the understanding of correlations between patient digital communication behavior, health states, and appropriate therapeutic interventions.

Fifth, the technology can provide technical solutions necessarily rooted in computer technology (e.g., utilizing computer models for selecting therapeutic interventions tailored to a patient health state inferred from digital communication behavior and/or sensor data; dynamically modifying a dynamic care plan based on user behavior data, etc.) to overcome issues specifically arising with computer technology (e.g., issues surrounding how to use a plethora of patient digital communication data, sensor data, and/or actively collected data to optimize selection and delivery of therapeutic interventions).

Sixth, the technology can leverage specialized computing devices (e.g., computing devices with mobility-related sensors, physical activity monitoring capabilities, and/or other non-generalized functionality) to collect specialized datasets for selecting and/or promoting therapeutic interventions in the form of a personalized, modifiable dynamic care plan.

The technology can, however, provide any other suitable benefit(s) in the context of using non-generalized computer systems for generating and/or administering a dynamic care plan for a patient.

### 3. Method.

As shown in FIGS. 1A-1B, a method 100 for digitally providing healthcare to a patient includes: receiving a log of use dataset associated with patient digital communication behavior at a mobile computing device, wherein the log of use dataset corresponds to a time period S110; receiving a supplementary dataset corresponding to the time period S115; receiving a survey response dataset from the patient, the survey response dataset corresponding to the time period S120; receiving a care provider dataset in association with the time period S125; selecting a therapeutic intervention from a set of therapeutic interventions, based on processing with at least one of the first log of use dataset, the supplementary dataset, the survey response dataset, and the care provider dataset S140; generating a dynamic care plan modifiable over a time period S150; promoting the therapeutic intervention according to the dynamic care plan S160. The method 100 can additionally or alternatively include dynamically modifying the dynamic care plan S170; and/or evaluating patient improvement S180.

### 3.1.A Passive Data—Receiving a Log of Use Dataset.

As shown in FIGS. 1A-1B, 2A-2B, and 3A, Block S110 recites: receiving a log of use dataset associated with patient digital communication behavior on a mobile computing device, wherein the log of use dataset corresponds to a time period, which functions to unobtrusively collect and/or retrieve mobility and communication-related data from a user's mobile computing device.

In relation to the log of use of the communication application, Block S110 is preferably implemented using a module of a processing system configured to interface with a native data collection application executing on a mobile computing device (e.g., smartphone, tablet, cardiovascular health monitoring device, cardiovascular health treatment device; personal data assistant, personal music player, vehicle, head-mounted wearable computing device, wrist-borne wearable computing device, etc.) of the user, in order to retrieve communication-related data pertaining to the user. As such, in one variation, a native application with data collection functions can be installed on the mobile computing device of the user (e.g., upon election of installation by the user, upon promotion of the user to the individual), can execute substantially continuously while the mobile computing device is in an active state (e.g., in use, in an on-state, in a sleep state, etc.), and can record communication parameters (e.g., communication times, durations, contact entities) of each inbound and/or outbound communication from the mobile computing device. In implementing Block S110, the mobile computing device can then upload this data to a database (e.g., remote server, cloud computing system, storage module), at a desired frequency (e.g., in near real-time, every hour, at the end of each day, etc.) to be accessed by the processing system. In one example of Block S110, the native data collection application can launch on the user's mobile computing device as a background process that gathers data from the user once the individual logs into an account (e.g., associated with a client application), where the data includes how and with what frequency the user interacts with and communicates with other individuals through phone calls, e-mail, instant messaging (e.g., at a particular client application, at a 3^{rd} party client application, etc.), an online social network, and/or any other suitable avenue of communication.

In relation to Block S110, a log of use dataset is preferably associated with a temporal indicator (e.g., time point, time window, time period, minute, hour, day, month, etc.) indicating when one or more digital communications occurred. However, a digital communication dataset can be distinct from temporal indicators. Receiving a log of use dataset can be performed before, during, in response to, and/or after selecting a therapeutic intervention S142, generating a dynamic care plan S144, promoting a therapeutic intervention S146, and/or any other suitable portion of the method 100. Collecting a log of use dataset can be performed during a same and/or overlapping time period as collecting another dataset (e.g., another log of use dataset, supplemental dataset, active dataset, etc.). Datasets corresponding to a same and/or overlapping time period can be used in selecting a personalized therapeutic intervention to be promoted to a patient during a time period (e.g., in real-time to address an emergency health state of the patient such as a suicidal episode), and/or after the time period (e.g., providing health tips over time to facilitate health education). However, Block S110 can be performed at any suitable time.

As such, in accessing the log of use of the communication application, Block S110 preferably enables collection of one or more of: phone call-related data (e.g., number of sent and/or received calls, call duration, call start and/or end time, location of individual before, during, and/or after a call, and number of and time points of missed or ignored calls); text messaging (e.g., SMS text messaging, messaging at a message platform within a client application, messaging with a health coach, etc.) data (e.g., number of messages sent and/or received, message length, message entry speed, delay between message completion time point and sending time point, message efficiency, message accuracy, time of sent and/or received messages, location of the individual when receiving and/or sending a message); data on textual messages sent through other communication venues (e.g., public and/or private textual messages sent to contacts of the user through an online social networking system, reviews of products, services, or businesses through an online ranking and/or review service, status updates, “likes” of content provided through an online social networking system), vocal and textual content (e.g., text and/or voice data that can be used to derive features indicative of negative or positive sentiments) and any other suitable type of data.

Additionally or alternatively, receiving a log of use dataset can be performed in any manner analogous to embodiments, variations, and examples of which are described in U.S. application Ser. No. 15/069,163, entitled “Method for Providing Patient Indications to an Entity” and filed on 14 Mar. 2016, which is herein incorporated in its entirety by this reference.

However, receiving a log of use dataset S110 can be performed in any suitable manner.

### 3.2 Passive Data—Receiving a Supplemental Dataset.

As shown in FIGS. 1A-1B, 2A-2B, and 3A, Block S115 recites: receiving a supplementary dataset (equivalently referred to herein as a supplemental dataset) associated with the time period, which functions to unobtrusively receive non-communication-related data from a patient's mobile computing device and/or other device configured to receive contextual data from the patient.

Block S115 can include receiving one or more of: location information, movement information (e.g., related to physical isolation, related to lethargy), device usage information (e.g., screen usage information related to disturbed sleep, restlessness, and/or interest in mobile device activities), and any other suitable information.

In variations, Block S115 can include receiving location information of the patient by way of one or more of: receiving a GPS location of the individual (e.g., from a GPS sensor within the mobile communication device of the patient), estimating the location of the patient through triangulation (e.g., triangulation of local cellular towers in communication with the mobile communication device), identifying a geo-located local Wi-Fi hotspot during a phone call, and in any other suitable manner. In applications, data received in Block S110 and S115 can be processed to track behavior characteristics of the patient, such as mobility, periods of isolation, quality of life (e.g., work-life balance based on time spent at specific locations), and any other location-derived behavior information. In an example, Block S115 can include receiving a mobility behavior supplementary dataset associated with a mobility-related sensor (e.g., single or multi-axis accelerometer, gyroscope, GPS module, gravity sensor, step counter sensor, step detector sensor, rotation sensor, location sensor, magnetic sensors, pressure sensors, etc.) of the mobile computing device (e.g., where the mobility supplementary dataset corresponds to the time period in which a log of use dataset was received in Block S110). In a specific example, Block S115 can include receiving a mobility behavior supplementary dataset including linear acceleration data along the x-, y-, and/or z-axis with a multi-axis accelerometer. In another specific example, Block S115 can include receiving a mobility behavior supplementary dataset including rate of rotation data around the x-, y-, and/or z-axis with a multi-axis gyroscope.

In additional or alternative variations, Block S115 can additionally or alternatively include receiving one or more of: physical activity- or physical action-related data (e.g., accelerometer data, gyroscope data, data from an M7 or M8 chip, Apple HealthKit data, etc.) of the patient, local environmental data (e.g., climate data, temperature data, light parameter data, etc.), nutrition or diet-related data (e.g., data from food establishment check-ins, data from spectrophotometric analysis, etc.) of the patient, biometric data (e.g., data recorded through sensors within the patient's mobile communication device, data recorded through a wearable or other peripheral device in communication with the patient's mobile communication device) of the patient, and any other suitable data. In examples, one or more of: a blood pressure sensor, and a pulse-oximeter sensor, and an activity tracker can transmit the individual's blood pressure, blood oxygen level, and exercise behavior to a mobile communication device of the individual and/or a processing subsystem implementing portions of the method 100, and Block S115 can include receiving this data to further augment analyses performed in Block S142.

In relation to a sensor signal processing module, Block S110 is preferably implemented using a module of a processing system configured to interface with a sensor signal processing module of a mobile computing device (e.g., smartphone, tablet, cardiovascular device, personal data assistant, personal music player, vehicle, head-mounted wearable computing device, wrist-borne wearable computing device, etc.) of a user, in order to retrieve data that can be used to assess mobility behavior of the user. In variations, the sensor signal processing module can receive signals derived from one or more of: an accelerometer, a gyroscope, a compass, a GPS module, a force sensor, and any other suitable sensing module that can produce signals indicative of user mobility (e.g., within his/her environment). In specific examples, accessing the sensor signal processor module in Block S110 can include accessing data derived from one or more of: an Apple M7 chip, an Apple M8 chip, a microprocessor of a mobile computing device, a storage unit of a mobile computing device, and any other specific sensor signal processor module. Additionally or alternatively, in Block S110, accessing data indicative of user mobility can occur by accessing information derived from other native applications (e.g., health monitoring applications, activity monitoring applications, etc.) executing on the mobile device of the user, for instance, as facilitated using an application programming interface (API). As such, data from the sensor signal processing module can be accessed indirectly through APIs associated with one or more other applications executing on a user's mobile device. In any of the above embodiments, variations, and examples, accessing a sensor signal processing module can be facilitated using a native application (e.g., a native application with input, output, and data collection functions) installed on the mobile computing device of the user, and/or in any other suitable manner.

In relation to Block S115, a supplemental dataset is preferably associated with a temporal indicator. For example, Block S115 can include receiving a supplementary dataset (e.g., a mobility behavior supplementary dataset) corresponding to a time period. In a variation, Block S115 can include receiving a supplementary dataset corresponding to a time period in which a cardiovascular treatment (e.g., a cardiovascular therapeutic intervention promoted in Block S146) was administered. For example, Block S115 can include receiving a supplementary dataset collected at a cardiovascular device, the supplementary dataset corresponding to a time period immediately following administration of the cardiovascular treatment. However, a supplemental dataset can be associated with any suitable temporal indicators, or can be distinct from temporal indicators.

Block S115 can include receiving supplemental data pertaining to the patient before, during, and/or after (or in the absence of) patient communication with another individual and/or computer network (e.g., as described in Block S110), selection of a therapeutic intervention (e.g., in Block S144), generation of a care plan (e.g., in Block S146), promotion of a therapeutic intervention (e.g., in Block S140), and/or any other suitable portion of the method 100.

Additionally or alternatively, receiving a supplemental dataset S115 can be performed in any manner analogous to embodiments, variations, and examples described in U.S. application Ser. No. 15/245,571, entitled “Method and System for Modeling Behavior and Heart Disease State” and filed on 24 Aug. 2016, which is herein incorporated in its entirety by this reference.

However, receiving a supplementary dataset S115 can be performed in any suitable manner.

### 3.3 Active Data—Receiving a Survey Response Dataset.

As shown in FIGS. 1A-1B, 2A-2B, 3A-3B, and 11A-11B, Block S120 recites: using the application, receiving a survey response dataset in association with a time period, which functions to receive active data provided by surveying the user and/or other suitable entity (e.g., a care provider, friends, family, etc.). Block S120 can additionally or alternatively function to administer surveys for collecting data that can be leveraged in generating and/or dynamically modifying a care plan. For example, evaluative surveys assessing a patient's feelings about a therapeutic intervention can be used in dynamically modifying a care plan to include similar therapeutic interventions (e.g., in response to positive patient evaluations) or dissimilar therapeutic interventions (e.g., in response negative patient evaluations). Block S120 thus enables generation of active data (e.g., data actively provided by a user) that can contribute to indication generation in subsequent blocks of the method 100.

Block S120 is preferably implemented at a module of the processing system described in relation to Block S110 above, but can additionally or alternatively be implemented at any other suitable system configured to receive survey data from one or more users. The survey response dataset can include interview and/or self-reported information from the user. Furthermore, the survey response dataset preferably includes quantitative data, but can additionally or alternatively include qualitative data pertaining to a disorder-related state of the user and corresponding to a set of time points of the time period. In relation to sensor-derived and communication-derived data received in Block S110, portions of the survey response dataset preferably correspond to a time period overlapping with the time period associated with the sensor-data and communication data; however, portions of the survey response dataset can alternatively correspond to time points outside of the time period associated with Block S110 (e.g., as in a pre-screening or a post-screening survey). Additionally or alternatively, Block S120 can include receiving clinical data (e.g., information gathered in a clinic or laboratory setting by a clinician).

In Block S120, time points of the time period can include uniformly or non-uniformly-spaced time points, and can be constrained within or extend beyond the time period of the log of use of the communication application of Block S110. As such, in variations, the time points of the time period can include regularly-spaced time points (e.g., time points spaced apart by an hour, by a day, by a week, by a month, etc.) with a suitable resolution for enabling detection of changes in a disorder-related state of the user. Additionally or alternatively, provision of a survey and/or reception of responses to a survey can be triggered upon detection of an event of the user (e.g., based upon data from sensors associated with the user, etc.) or any other suitable change in disorder-related state of the user. Furthermore, the same survey(s) can be provided to the user for all time points associated with the time period; however, in alternative variations, the same survey(s) may not be provided to the user for at least one time point associated with the time period.

In variations of Block S120, the survey response dataset can include responses to one or more surveys configured to assess severity of one or more of: depression, pain, rheumatoid disorders, psychosis (e.g., along a schizophrenia spectrum), cardiovascular disease, sleep disorders, and any other suitable condition or type of condition. Furthermore, the surveys can be configured to transform qualitative information capturing a user's state into quantitative data according to a response-scoring algorithm. In examples, the survey(s) implemented in Block S120 can be derived from depression-assessment surveys including one or more of: a Hamilton Rating Scale for Depression (HAM-D); the User Health Questionnaire (PHQ-9, PHQ-2) for screening, monitoring, and measuring depression severity according to Diagnostic and Statistical Manual (DSM) criteria for depression; the World Health Organization (WHO-5) quality of life assessment; the User Activation Measure (PAM) self-management; and any other suitable depression-assessment survey.

Additionally or alternatively, the survey(s) implemented in Block S120 can be derived from pain-assessment surveys including one or more of: a Wong-Baker FACES pain rating scale (with pain rated on a scale from 0-5, 5 being the most severe); a pain visual analog scale (VAS); a pain numeric rating scale (NRS); a verbal pain intensity scale; a brief pain inventory (BPI) tool; a rheumatic disease specific pain scale (DSPI) scored according to sum(X*Y), where X is the pain level on a 0-10 scale and Y is the percentage of this pain level in a given rheumatic disease group; an Osteoarthritis Research. Society International-Outcome Measures in Rheumatoid Arthritis Clinical Trials (OARSI-OMERACT) tool; a survey describing pain location (e.g., with respect to a specific joint, with respect to location within a specific joint); a survey describing pain type (e.g., sharp pain, dull pain, etc.); a survey identifying pain cause (e.g., injury, aging, degeneration, etc.), a survey identifying pain frequency (e.g., with regard to regularity), a survey identifying patterns in pain (e.g., time of pain, weather-related pain, time-of-day-related pain, temperature-related pain, etc.), and any other suitable pain-related survey.

Additionally or alternatively, the survey(s) implemented in Block S120 can be derived from daily functioning and/or activity-assessment surveys including one or more of: a physical activity scale (PAS) survey; a PAS-II survey; a Health Assessment Questionnaire (HAQ, HAQ-II) with scores ranging from 0-3 (with 3 being most severe) a disease activity index (DAI) tool; and any other activity assessment tool. Additionally or alternatively, in examples, the set of symptom-assessment surveys can include surveys focused on symptom exhibition and severity assessment as derived from one or more of: a routine assessment of user index data tool; a rheumatic arthritis disease activity score (DAS) survey; an arthritis impact measurement scale (AIMS); a British Isles Lupus Assessment Group (BILAG) tool; a systemic lupus erythematosus (SLE) activity questionnaire; an SLE symptom scale survey; and any other suitable survey or tool for assessing symptom exhibition and severity.

Additionally or alternatively, the survey(s) implemented in Block S120 can be derived from psychiatric state assessment surveys including one or more of a Brief Psychiatric Rating Scale (i.e., a 16-18 item survey of psychiatric symptom constructs including somatic concern, anxiety, emotional withdrawal, conceptual disorganization, guilt feelings, tension, mannerisms and posturing, grandiosity, depressive mood, hostility, suspiciousness, hallucinatory behavior, motor retardation, uncooperativeness, unusual thought content, blunted affect, excitement, and disorientation, first published in 1962); a Clinical Global Impression (CGI) rating scale; a Dimensions of Psychosis Symptom Severity scale provided by the American Psychiatric Association; a Global Functioning Role (GFR) survey for phases of Schizophrenia; a Global Functioning Social (GFS) survey for phases of Schizophrenia; a Community Assessment of Psychic Experiences (CAPE) derived survey; a Scale for the Assessment of Positive Symptoms (SAPS) derived survey for delusional behavior, hallucinatory behavior, and/or disorganized speech behavior; and any other suitable tool or survey for assessment of a disorder-related state.

Additionally or alternatively, other survey responses received in Block S120 can include one or more of: a demographic survey that receives demographic information of the user; a medication adherence survey (for users taking medication for a psychotic disorder); a mood survey; and a social contact survey (e.g., covering questions regarding aspects of the user's contact with others). However, the set of surveys can include any other suitable surveys configured to assess mental states of the user, or adaptations thereof. As such, the survey response dataset can include quantitative scores of the user for one or more subsets of surveys for each of a set of time points of the time period.

In an example of Block S120, the survey dataset includes biweekly responses (e.g., for a period of 6 months) to the PHQ-9 survey, biweekly responses (e.g., for a period of 6 months) to the WHO-5 survey in alternation with the PHQ-9 survey, responses to the PAM assessment at an initial time point, at an intermediate time point (e.g., l-month time point), and at a termination time point, responses to the HAM-D assessment at an initial time point and a termination time point, biweekly response to a recent care survey, daily responses to a mood survey, and twice-per-week responses to a medication adherence survey.

In some variations, Block S120 can further include facilitating automatic provision of at least one survey at the mobile computing device(s) of the user(s). As such, responses to one or more surveys can be provided by user input at an electronic device (e.g., a mobile computing device of the user), or automatically detected from user activity (e.g., using suitable sensors). Additionally or alternatively, provision of at least one survey can be performed manually by an entity associated with a user or received as derived from clinical data, with data generated from the survey(s) received in Block S120 by manual input. Additionally or alternatively, provision of at least one survey and/or reception of responses to the survey can be guided by way of an application executing at a device (e.g., mobile device, tablet) of a caretaker of the user and/or the user, where the application provides instruction (e.g., in an audio format, in a graphic format, in a text-based format, etc.) for providing the survey or the responses to the survey. Block S120 can, however, be implemented in any other suitable manner (e.g., by verbal communication over the phone, by verbal communication face-to-face, etc.).

However, receiving a survey dataset S120 can be performed in any suitable manner.

### 3.4 Active Data—Receiving a Care Provider Dataset.

As shown in FIGS. 1A-1B, 2A, and 3A-3B, Block S125 recites: receiving a care provider dataset in association with a time period, which functions to receive active data provided in association with a care provider, for use in selecting and/or promoting a therapeutic intervention, generating and/or dynamically modifying a dynamic care plan, and/or any other purpose.

In relation to Block S125, a care provider can include any one or more of: a psychiatrist, physician, healthcare professional, health coach, therapist, guardian, friend, and/or any suitable provider of care for one or more patients.

A care provider dataset preferably includes care provider observations, assessments and/or insights regarding interactions (e.g., textual interactions, audio, video, etc.) with a patient, but can include any suitable data in relation to one or more patients. Interactions with a patient can be through any one or more of: in-person communication (e.g., a scheduled appointment), digital communication (e.g., test messaging communication), and/or any suitable venue.

For Block S125, care provider data can be collected through a web interface, an application executing on a mobile computing device (e.g., a care provider device), and/or any suitable venue. For example, Block S125 can include receiving a care provider dataset in response to prompting a care provider to provide a care provider input (e.g., at a web interface displaying patient information including a patient improvement evaluation, etc.). Care provider data is preferably collected, processed, and/or leveraged through the generation and administration of a dynamic care plan. For example, the method 100 can include receiving a first care provider dataset during a first time period; generating a dynamic care plan based on the first care provider dataset, during a second time period subsequent the first time period; receiving a second care provider dataset during a third time period subsequent a second time period; and dynamically modifying the dynamic care plan based on the second care provider dataset, during a fourth time period subsequent the third time period. However, receiving a care provider dataset can be performed at any suitable time.

Additionally or alternatively, receiving a care provider dataset can be performed in any manner analogous to embodiments, variations, and examples described in U.S. application Ser. No. 15/005,923, entitled “Method for Providing Therapy to an Individual” and filed on 25 Jan. 2016, which is herein incorporated in its entirety by this reference.

### 3.5 Processing Data.

Block S130 recites: processing at least one of a log of use dataset, a supplemental dataset, and/or an active dataset (e.g., a survey dataset, input from a care provider, etc.). Block S130 functions to process data collected at least in one of Block S110, S115, and S120 for use in subsequent portions of the method 100. Block S130 can additionally or alternatively include generating a behavioral dataset S132.

Processing data can include any one or more of: extracting features, performing pattern recognition on data, fusing data from multiple sources (e.g., from patients, from care providers, active data, passive data, etc.), combination of values (e.g., combining mobility behavior data collected from different mobility-related sensors, etc.), compression, conversion (e.g., digital-to-analog conversion, analog-to-digital conversion), wave modulation, normalization, filtering, noise reduction, smoothing, model fitting, transformations, mathematical operations (e.g., derivatives, moving averages, etc.), multiplexing, demultiplexing, and/or any other suitable processing operations

However, processing a dataset S130 can be performed in any suitable manner.

### 3.5.A Generating a Behavioral Dataset.

Block S130 can optionally include Block S132, which recites: generating a behavioral dataset derived from the sensor signal processor module and the log of use, associated with the time period and derived from passive behavior. Block S132 functions to generate a behavioral dataset based upon unobtrusively collected data from the mobile computing device and/or other device associated with the individual, where the device is configured to receive contextual data pertaining to mobility-related behaviors of the individual. The behavioral dataset can thus be subject to clinically-informed behavioral rules (e.g., determined using heuristics), which can contribute to indication generation in subsequent blocks of the method 100. Block S132 can include reception of non-communication-related data pertaining to the individual before, during, and/or after (or in the absence of) communication with another individual (e.g., a phone call) and/or computer network (e.g., an online social networking application), as described above in relation to Block S110. Block S132 can include receiving one or more of: location information, motion information (e.g., related to physical isolation, related to lethargy), device usage information (e.g., screen usage information related to disturbed sleep, restlessness, interest in mobile device activities, usage of mobile device applications, data load attributed to each of a set of mobile device applications), and any other suitable information. The behavioral dataset generated in Block S132 is preferably derived from sensors on-board the mobile computing device (e.g., GPS sensors, accelerometers, gyroscopes, M7 chips, M8 chips) and/or sensors in communication with the mobile computing device (e.g., sensors of devices configured to sync with the mobile computing device), as described in relation to Block S110 above; however, the supplementary dataset can alternatively be derived from any other suitable system.

In variations, Block S132 can include generating location-based behavioral information of the user by way of one or more of: receiving a GPS location of the user (e.g., from a GPS sensor on-board the mobile computing device of the individual), estimating the location of the user through triangulation of local cellular towers in communication with the mobile computing device, identifying a geo-located local Wi-Fi hotspot during a phone call, and any other suitable method for location approximation/identification. In applications, data received in Block S110 and processed in S132 can be used to track behaviors of the user, such as behaviors indicative of mobility, behaviors indicative of periods of isolation, behaviors indicative of quality of life (e.g., work-life balance based on time spent at specific locations), and any other location-derived behavior information. As such, data from Blocks S110 and S132 can be merged to track the individual's mobility during a communication, in the relation to therapeutic intervention predictive models and/or analyses generated in subsequent blocks of the method 100. In variations, Block S132 can additionally or alternatively include generating mobile device usage data, including data indicative of screen unlocks and mobile application usage (e.g., by retrieving usage information from mobile operating system logs, by retrieving usage information from a task manager on a mobile computing device, etc.). Blocks S132 and/or S110 can therefore provide data that facilitates tracking of variations and periods of activity/inactivity for a user through automatically collected data (e.g., from the user's mobile computing device), in order to enable identification of periods of activity and inactivity of the user (e.g., periods when the user was hyperactive on the device or not asleep).

In additional variations, Block S132 can additionally or alternatively include generating one or more of: physical activity- or physical action-related data (e.g., accelerometer and gyroscope data) for the user, local environmental data (e.g., climate data, temperature data, light parameter data, etc.) associated with the user, nutrition or diet-related data (e.g., data from food establishment check-ins, data from spectrophotometric analysis, etc.) associated with the user, biometric data (e.g., data recorded through sensors within the user's mobile computing device, data recorded through a wearable or other peripheral device in communication with the user's mobile computing device), and any other suitable data. In examples, one or more of: an activity monitor (e.g., Apple Watch, FitBit device, etc.), a blood pressure sensor, and a pulse-oximeter sensor can transmit the individual's activity behavior, blood pressure, and/or blood oxygen level to a mobile computing device of the user and/or a processing system implementing portions of the method 100, and Block S132 can include processing this data to further augment models generated in Block S142.

In relation to receiving and processing data, Blocks S132, S110, S115, and/or S120 can additionally or alternatively include receiving data pertaining to individuals in contact with the user during the period of time, such that data from the user and data from one or more individuals in communication with the user are received (e.g., using information from an analogous application executing on the electronic device(s) of the individual(s) in communication with the individual). As such, Blocks S132, S110, S115, and/or S110 can provide a holistic view that aggregates communication behavior data and contextual data of two sides of a communication involving the user. In examples, such data can include one or more of: an associated individual's location during a phone call with the user, the associated individual's phone number, the associated individual's length of acquaintance with the user, and the associated individual's relationship to the user (e.g., top contact, spouse, family member, friend, coworker, business associate, etc.).

However, generating a behavioral dataset S130 can be performed in any suitable manner.

### 3.6 Selecting a Therapeutic Intervention.

As shown in FIGS. 1A-1B, 2A-2B, and 3A, Block S140 recites: selecting a therapeutic intervention from a set of therapeutic interventions, which functions to identify a suitable therapeutic intervention for the user. Block S140 can additionally or alternatively include generating a therapeutic intervention predictive model S142, generating a comparison between a dataset and a threshold condition S144, and/or determining a patient health state S146.

In relation to Block S140, a therapeutic intervention type preferably falls into one of a set of forms that are conducive for delivery to the user in an efficient manner in Block S160. In variations, types of therapeutic interventions can include any one or more of: health improving tips and health state information associated with one or more therapeutic intervention categories (e.g., motivational, psychoeducational, cognitive behavioral, biological, physical, mindfulness-related, relaxation-related, dialectical behavioral, acceptance-related, commitment-related, skill-based, empathy, etc.); media (e.g., images, graphics, audio, video, virtual reality, and/or other types of media configured to improve a patient health state); mental health exercises (e.g., mindfulness activities, breathing activities, breath awareness activities, savoring activities, etc.); interventions associated with therapeutic intervention categories for restlessness, lack of focus, sleep, abnormal communication behavior, and isolation; health improving kits (e.g., with media/regimens) for improving health according to one or more therapeutic intervention categories; medications/therapeutic substances; enabling the user to communicate with an entity that provides therapeutic communication (e.g., a care provider such as a therapy providing entity, nurse, psychologist, psychiatrist, physical therapist, etc.); and/or any other suitable therapeutic intervention type.

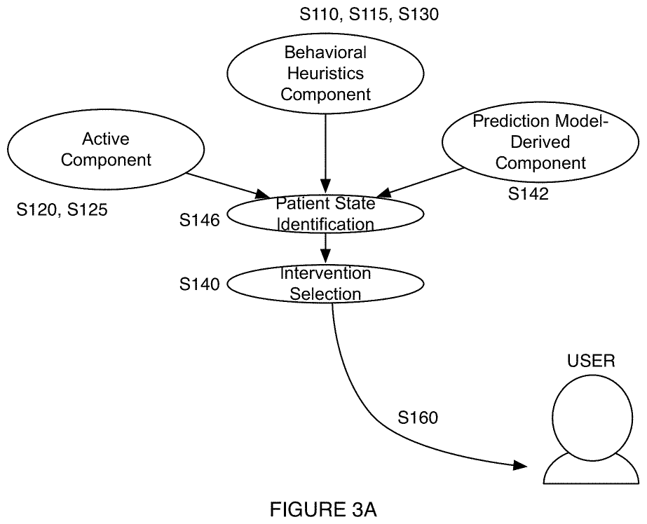

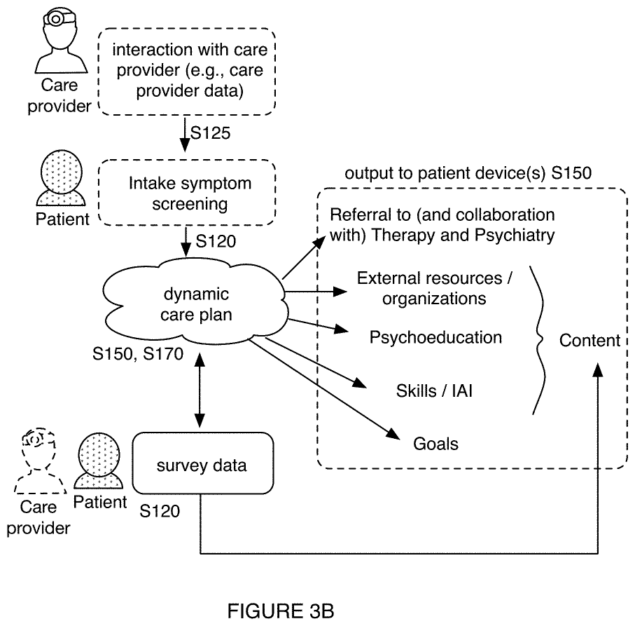

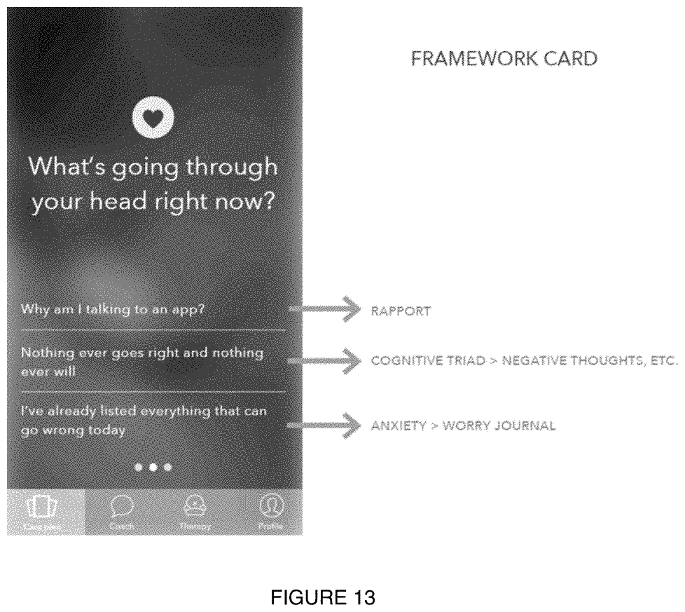

As shown in FIGS. 3 and 13, Block S140 preferably includes selecting a therapeutic intervention based on at least one or more of: a log of use dataset factor, a supplementary dataset (e.g., a mobility behavior supplementary dataset) factor, and an active dataset (e.g., a survey dataset, a care provider dataset, etc.) factor. For example, a dynamic care plan calibration survey can be presented, where patient responses can be mapped to patient health states and/or appropriate therapeutic interventions. In a specific example, selecting a therapeutic intervention (and/or generating a dynamic care plan) can be based on active data collected from engaging the patient in a digital calibration conversation (e.g., with a chat bot, with a virtual assistant, with a care provider). The digital calibration conversation is preferably automated (e.g., scripted with a conversational, natural, user-centric tone) but can additionally or alternatively be with another individual. In another example, Block S140 can extracting a mobility behavior characteristic (e.g., average patient travel radius per day) from a mobility behavior supplemental dataset; and, in response to the mobility behavior characteristic below a threshold (e.g., a patient travel radius threshold, below which indicates stress, anxiety, or unhappiness), selecting a therapeutic intervention (e.g., prompting a patient to play an augmented reality game requiring the patient to take a walk outside. Additionally or alternatively, selecting a therapeutic intervention S140 can be based on an identified patient health state (e.g., as described in Block S146). For example, the method 100 can include generating a therapeutic intervention predictive model from at least one of a log of use dataset and a mobility behavior supplementary dataset; determining a health state of the patient based upon at least one of an output of the therapeutic intervention predictive model, the log of use dataset, and the mobility behavior supplementary dataset; and selecting the therapeutic intervention from the set of therapeutic interventions, based on the health state of the patient. However, selecting a therapeutic intervention S140 can be based on any suitable data.

Block S140 can include selecting a therapeutic intervention as an output from processing acquired data with a therapeutic intervention predictive model (e.g., described in Block S142), comparisons with thresholds (e.g., described in Block S144), associations between therapeutic interventions and patient health states (e.g., described in Block S146), and/or other suitable approaches. Selecting the therapeutic intervention can be performed by an entity (e.g., computing system, person) other than the user; however, in some variations, the therapeutic intervention can be selected by the user (upon receiving an input provided by the user at his/her mobile computing device). Block S140 preferably selects a therapeutic intervention type and/or category from a previously identified set of therapeutic intervention types and/or categories known to positively affect users in an adverse state of health similar or identical to the state of the user determined in Block S146. For example, the method 100 can include generating a patient profile (e.g., describing digital communication behaviors, sensor-related behavior, active data, etc.); identifying a reference profile (e.g., a different patient's profile, a curated reference profile, an automatically generated reference profile, etc.) with the greatest similarity to the patient profile, where the reference profile is associated with one or more therapeutic interventions and/or categories (e.g., known to positively affect patients with similar profiles); and promoting the one or more therapeutic interventions and/or categories to the patient.

However, Block S140 can additionally or alternatively select or determine improvised new therapeutic intervention categories and therapeutic intervention types specific to the state of the user, which can be experimentally used to determine appropriate therapeutic interventions for other users in a state similar to that experienced by the user. As such, Block S140 can select static therapeutic interventions from a pre-identified set of therapeutic interventions, or can adaptively form new therapeutic interventions for provision to a user. Processing of responses to static and/or new therapeutic interventions across a population of users (e.g., according to correlational studies, according to machine learning algorithms, etc.), using methods similar to those described above, can further be used to improve efficacy and appropriateness of therapeutic interventions provided to users, as more data is collected (i.e., to provide data-driven therapeutic interventions). Additionally or alternatively, Block S140 can select multiple therapeutic interventions and therapeutic intervention types to provide a combinatorial therapeutic intervention to the user in Block S160.

In variations of Block S140, therapeutic interventions can be characterized by one or more therapeutic intervention categories. The therapeutic intervention category is preferably selected based upon identification of the type of health condition (e.g., a psychological disorder, a condition of depression, a pain-related condition, a sleep-related condition, a cardiovascular disease related condition, etc.) associated with the state of the user in Block S146. As such, in variations, the therapeutic intervention category can be specifically defined in relation to one or more of: a psychological state of the user, a depressive state of the user, a pain-related state of the user, a sleep-related state of the user, a cardiovascular disease-related state of the user, and any other suitable state of the user.

As shown in FIGS. 15-16, for Block S140, in variations of therapeutic intervention categories related to one or more of psychosis and depression, the therapeutic intervention categories can be selected from: a psychiatric management category (e.g., which includes therapeutic intervention types associated with education of the patient, education of acquaintances of the patient, forming alliances, providing support groups, etc.); a pharmacotherapeutic category (e.g., which includes therapeutic intervention types associated with antipsychotic medications, benzodiazepines, antidepressants, mood stabilizers, beta blockers); a cognitive behavioral therapy (CBT) category; a dialectical behavioral therapy (DBT) category (e.g., with therapeutic intervention types for treating borderline personality disorders); an acceptance and commitment therapy (ACT) category; an educational category (e.g., for therapeutic interventions focusing on educating the patient); a skill-based category (e.g., for therapeutic interventions aimed at developing patient skills in managing health); an empathy-focused category (e.g., for therapeutic interventions focused on empathizing with a patient health state and/or developing a patient's empathy); a practice-based category (e.g., exercises for practicing health skills and/or techniques); a take-away category (e.g., for summarizing information and providing take-home points); an interpersonal therapy category (e.g., with therapeutic intervention types associated with regaining control of mood); a problem solving therapy category; a psychodynamic psychotherapy category (e.g., with therapeutic intervention types associated with uncovering unconscious aspects of a person's psyche); a psychosocial therapeutic intervention category (e.g., with therapeutic interventions associated with improving a user's intersocial behavior); a weight management therapeutic intervention category (e.g., with therapeutic intervention types associated with preventing adverse weight-related side effects due to medications); a biosignal-related category (e.g., monitoring biosignals at a biosignal detector, electroconvulsive therapy, etc.); and any other suitable therapy category.

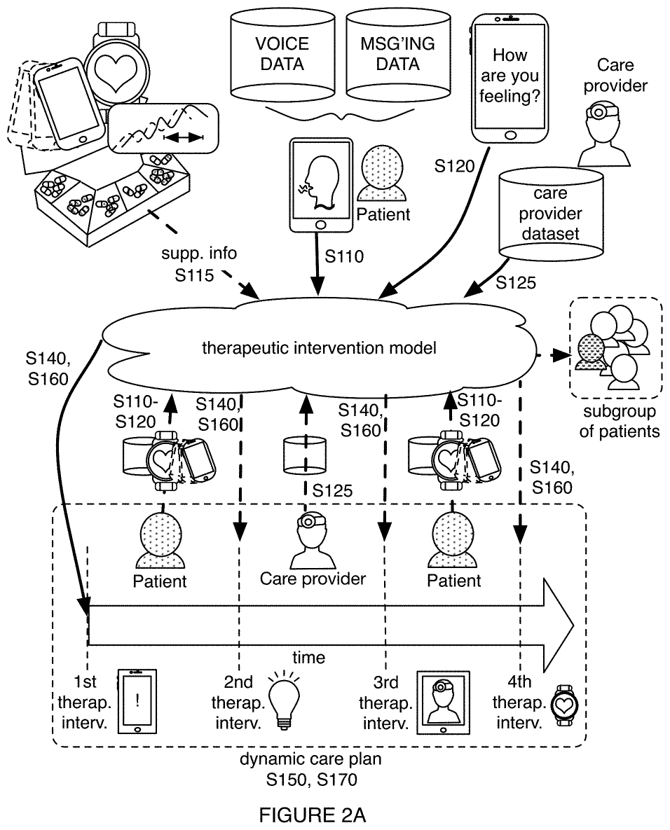

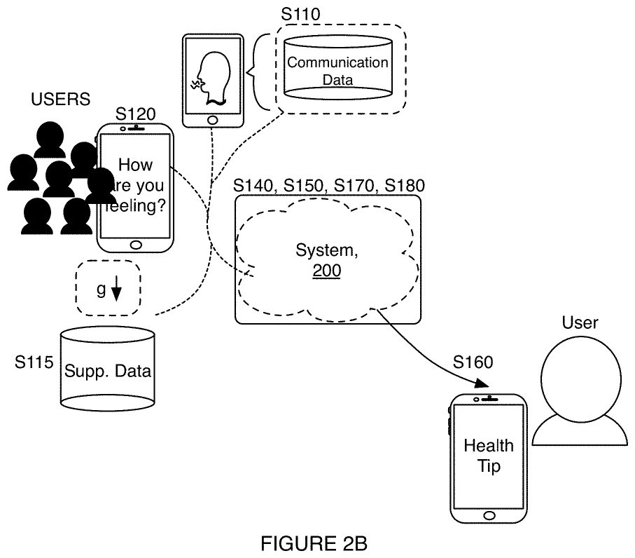

In a variation, as shown in FIG. 2A, Block S140 can include selecting a therapeutic intervention prompting a patient interaction with the therapeutic intervention. Therapeutic interventions encouraging patient interaction can improve patient engagement, adherence, and overall health state. Patient interactions can include any one or more of: inputs for exercises (e.g., selecting images representing patient mood; selecting a duration for a breathing exercise; choosing an answer for CBT educational games, etc.), inputs facilitating promotion of the therapeutic intervention (e.g., adjusting the volume on a soothing music audio therapy, pressing play on a video configured to incite happiness, etc.), therapeutic intervention evaluations (e.g., patient feedback regarding the therapeutic intervention, etc.), patient inputs at different patient devices (e.g., receiving data indicating patient inputs at a cardiovascular device to initiate heart rate monitoring, etc.), and/or any other suitable patient action in relation to a therapeutic intervention. Patient interactions can include patient interaction parameters such as temporal parameters (e.g., duration of interaction with a therapeutic intervention, frequency, time of day, etc.), parameters related to datasets from Blocks S110-S132 (e.g., a high level of digital communication prior to receiving the patient interaction with the therapeutic intervention; patient location during a patient interaction; heart rate of patient during a patient interaction; survey data indicating a stressed health state during a time period including the patient interaction, etc.), and/or any other suitable parameters. Patient interactions with a therapeutic intervention can be leveraged in selecting other therapeutic interventions (e.g., updating therapeutic intervention models, reference profiles, threshold conditions, etc.), generating a dynamic care plan (e.g., analyzing patient interaction with a given type of therapeutic interaction to infer when and/or how to administer future therapeutic interventions of the same type, etc.), dynamically adjusting a care plan (e.g., modifying therapeutic interventions to include easier educational exercises in response to a patient consistently choosing wrong answers in educational games, etc.), and/or performing other portions of the method 100.

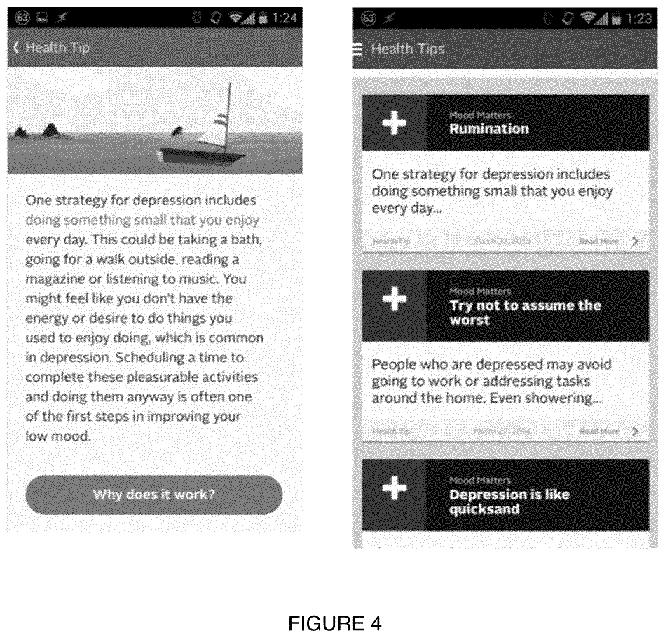

In another variation, as shown in FIGS. 4 and 12A-12B, the selected therapeutic intervention type can include one or more health tips and health state information associated with one or more therapeutic intervention categories (e.g., motivational, psychoeducational, cognitive behavioral, biological, physical, mindfulness-related, relaxation-related, dialectical behavioral, acceptance-related, commitment-related, etc.). In the example, the health tip is configured to provide the following information to a user: “One strategy for depression includes doing something small that you enjoy every day. This could be taking a bath, going for a walk outside, reading a magazine or listening to music. You might feel like you don't have the energy or desire to do things you used to enjoy doing, which is common in depression. Scheduling a time to complete these pleasurable activities and doing them anyway is often one of the first steps in improving your low mood”. The information in the health tip is configured to induce behavioral activation, which can be as effective as antidepressant medication in treating depression, and can be superior in treating more severe presentations of depression.

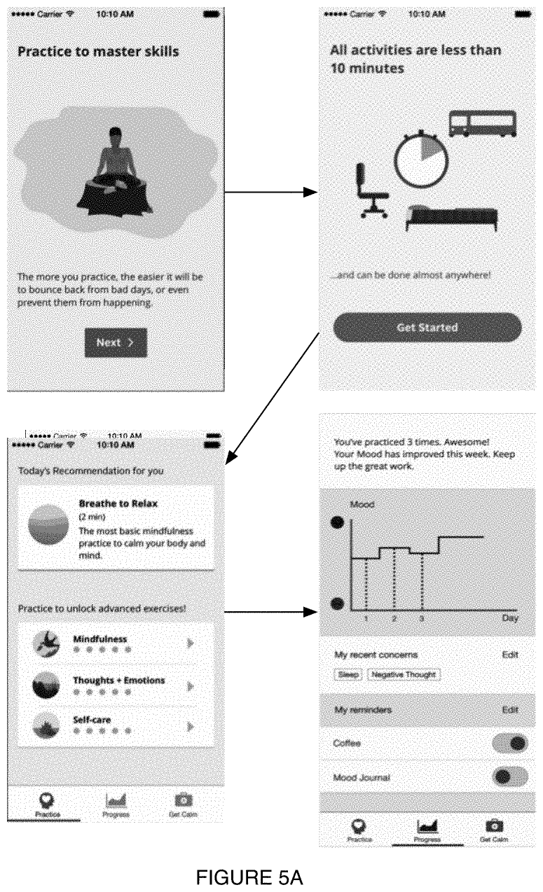

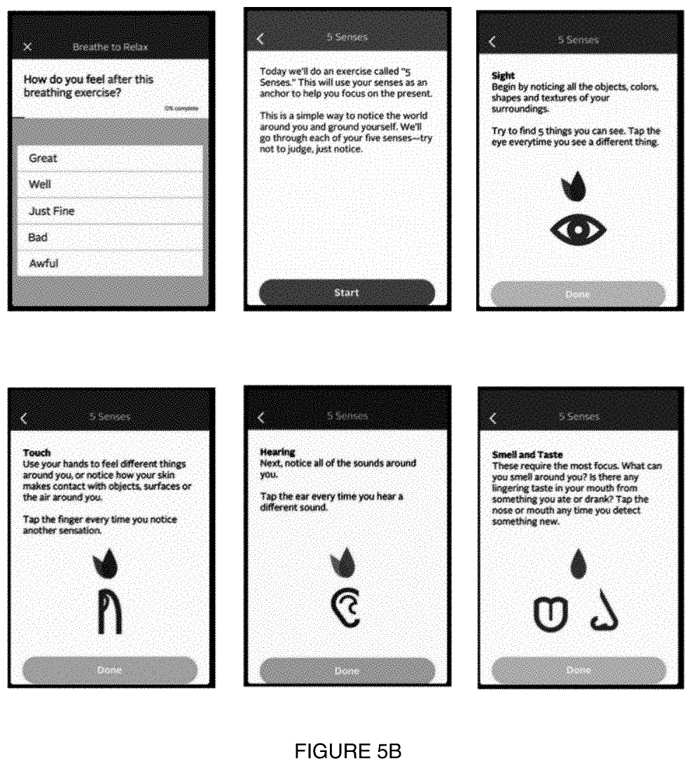

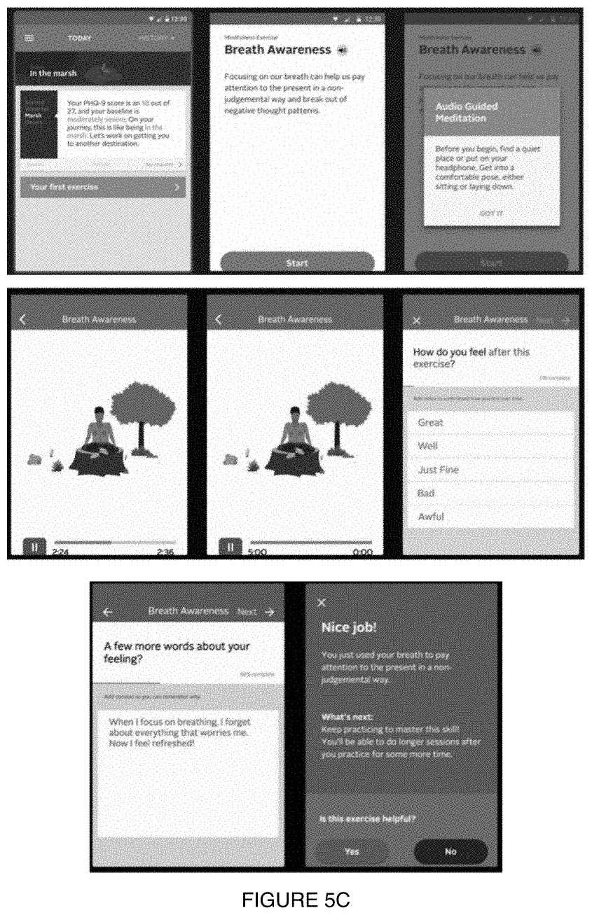

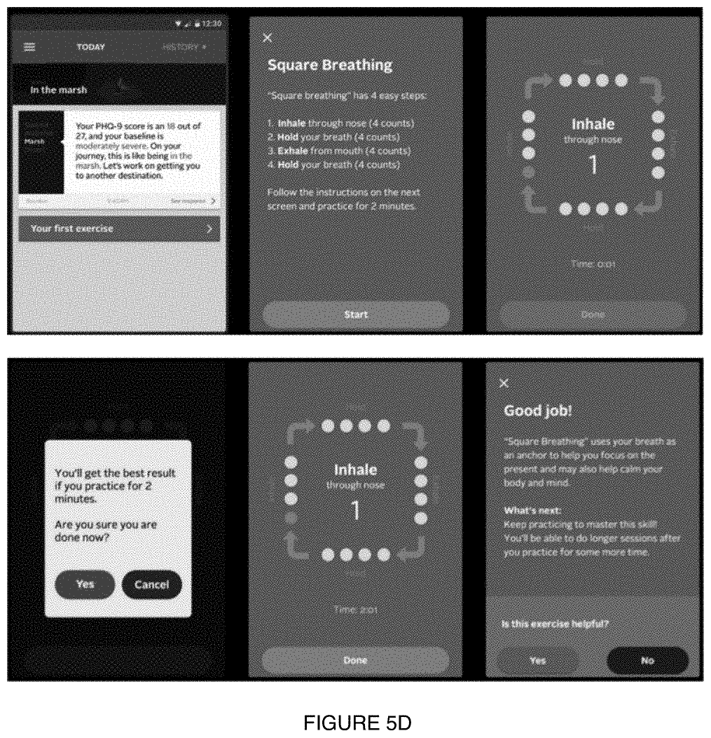

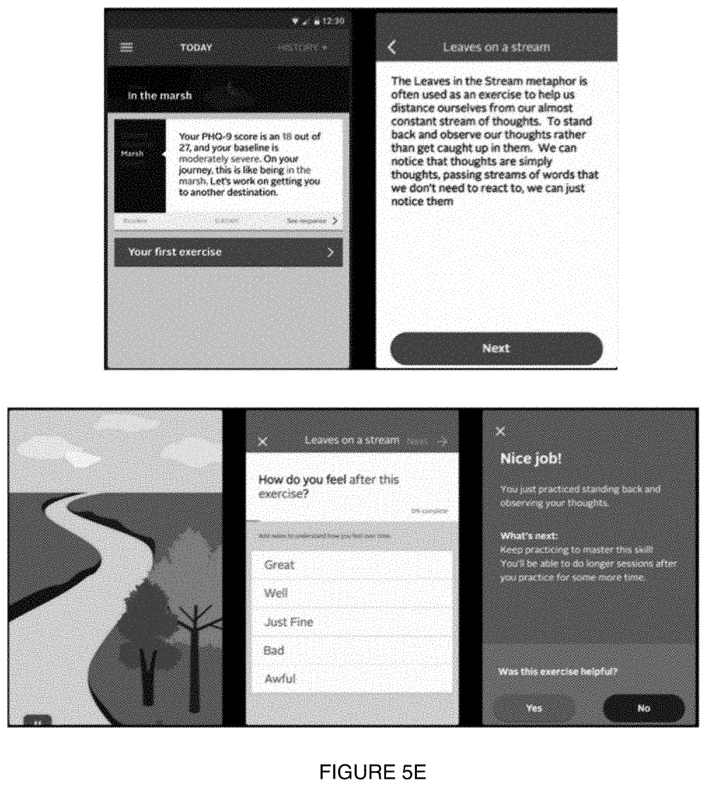

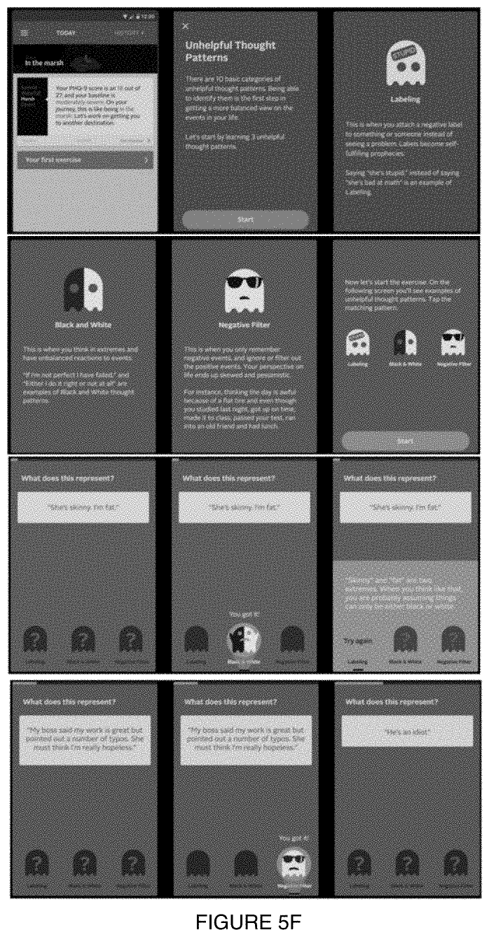

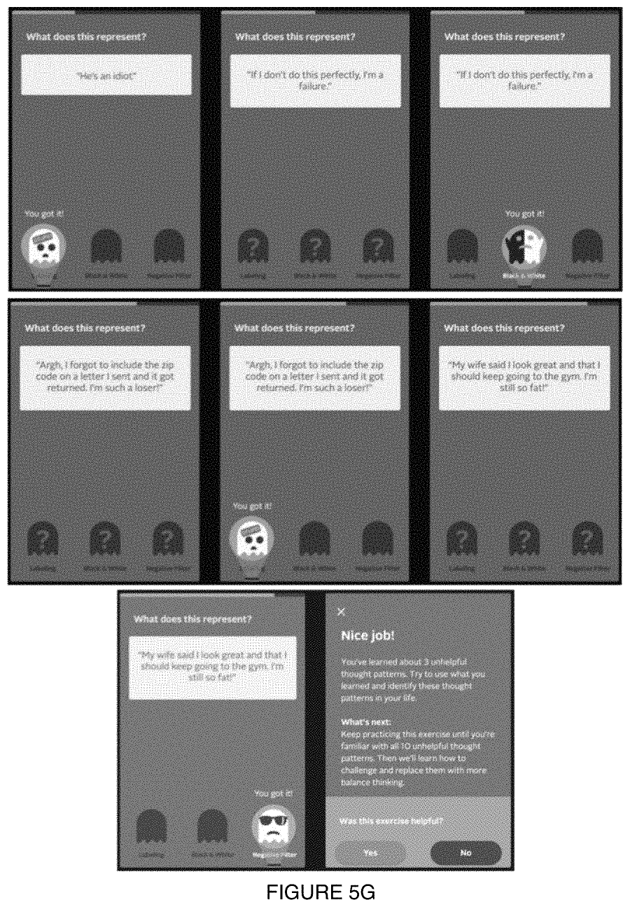

In another variation, as shown in FIG. 5A, the therapeutic intervention can include a daily (or with any other frequency) check in (e.g., a survey that guides the user in helping them be mindful of their state, to be positive when things are going well, etc.). The daily check in can also be associated with one or more activities to improve state through mindfulness activities that allow the user to focus on the present in a nonjudgmental way, by prompting the user to use his/her senses (e.g., sight, hearing, touch, smell, taste) to ground himself/herself. In this example, the therapeutic intervention can be configured to guide the user through one of a set of activities (e.g., breathing activity, mindfulness activity, self-care activity, emotion control activity, etc.), where one or more activities can be unlocked based upon achievement of other activities by the user. For instance, in an example mindfulness activity, as shown in FIG. 5C, the therapeutic intervention can be configured to guide the user to notice objects and their features within the user's surroundings, to hear all of the sounds in the user's environment, and to recognize the texture, smells, and tastes of things in the user's environment.

In a first example of a breathing activity, as shown in FIG. 5C, the user can be guided to focus on his/her breath (e.g., in relation to a high PHQ-9 score) with audio-guided medication (e.g., using speaker modules of a mobile computing device executing an associated application). In a second example of a breathing activity, as shown in FIG. 5D, the user can be guided through a “square breathing” activity, where the user performs a sequence of breathing behaviors (e.g., inhalation, breath holding, exhalation, etc.) for relaxation.

In an example of a thought control activity, as shown in FIG. 5E, the user can be guided to distance him/herself from a constant stream of thoughts (e.g., in relation to a high PHQ-9 score), such that the user learns to observe thoughts without immediately reacting to them. In more detail, as shown in FIG. 5E, the user can be provided with a rendering of a stream, along with audio (e.g., using display and speaker modules of a mobile computing device executing an associated application), to facilitate thought distancing behavior by the user.

In another example of a thoughts and emotion control activity, as shown in FIGS. 5F-5G, the user can be educated (e.g., using text and images rendered at a display) about different types of unhelpful thoughts (e.g., labeling, black and white thoughts, negative filters), and guided through an exercise where he/she is trained to identify different examples of different unhelpful thoughts. The therapeutic interventions can thus be configured to allow the user to progress along a certain health state (e.g., mood), as a motivational factor.

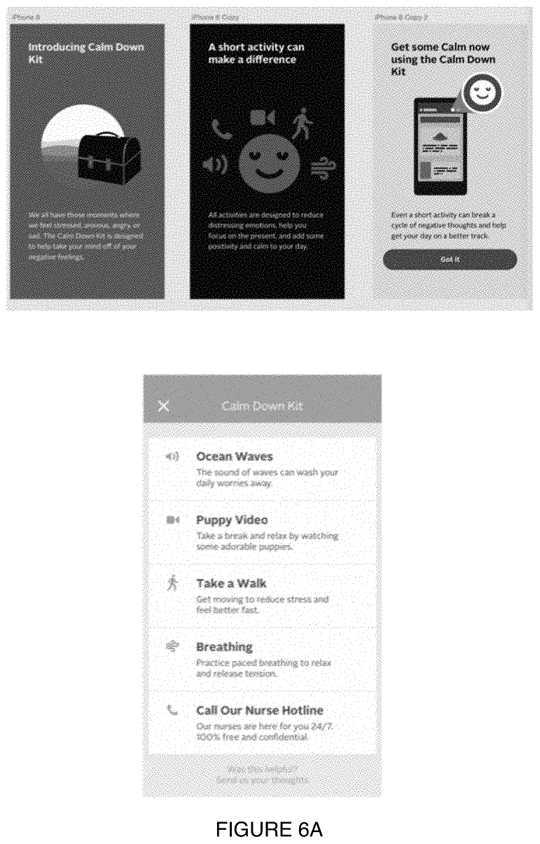

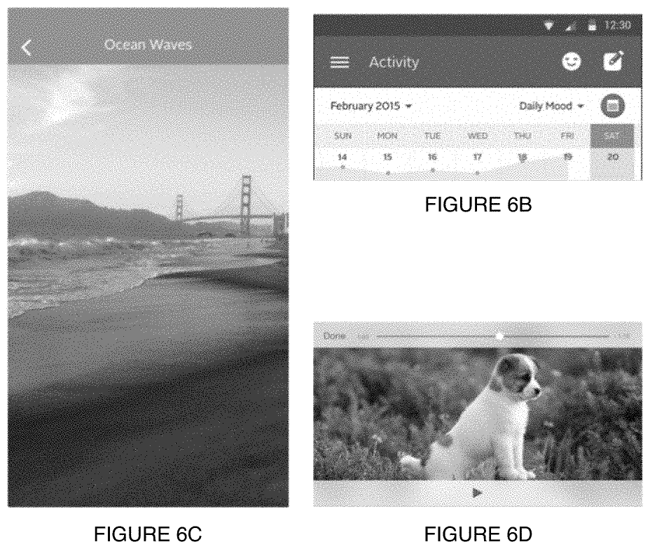

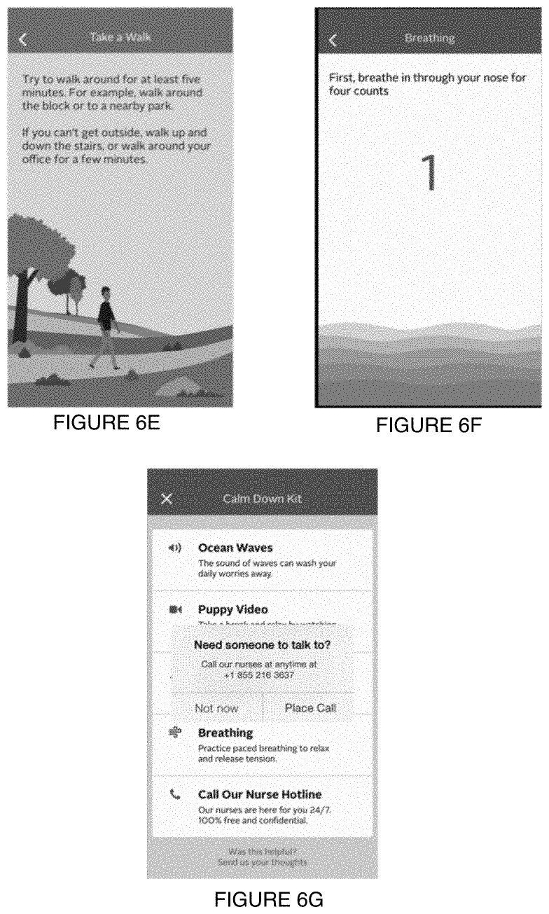

In another variation, as shown in FIGS. 6A-6G, the therapeutic intervention selected in Block S140 can include a relaxation kit that provides the user with media and/or activities that help the user with immediate support for their condition (e.g., to calm down for a panic attack). In the example, the relaxation kit can allow the user to select audio media (e.g., of ocean waves) as shown in FIG. 6C, video media (e.g., of a puppy) as shown in FIG. 6D, a physical activity (e.g., walking) as shown in FIG. 6E, a breathing activity as shown in FIG. 6F, and an option to reach out to a nurse for purposes of rectifying or resolving an adverse state, as shown in FIG. 6G. Additionally or alternatively, options to reach out to a therapeutic entity (or any other aspect of the relaxation kit) can be provided outside of the relaxation kit, in variations of this example. Furthermore, the relaxation kit of the therapeutic intervention can provide the user with a calendar and an option to make a journal entry for purposes of reflection, as shown in FIG. 6B, in order to allow the user to personally track his/her progress over time. In prompting the user to generate journal entries, the relaxation kit can provide the following prompt to the user: “Sometimes when depressed it can be difficult to remember things that are going well, or things for which you're grateful. For the next two weeks, spend a few minutes at the end of the day writing down five things for which you are thankful or grateful. Research has shown that writing down five things for which you are grateful every day improves positive emotions, well-being, and sleep in as little as two weeks.” Additionally or alternatively, Block S140 can include providing a user with an option to select media and/or an activity to include in the relaxation kit. For example, a user can add image media of their pet to a relaxation kit accessible at any time. However, the relaxation kit can include any other suitable modules and be configured to improve health of the user in any other suitable manner.

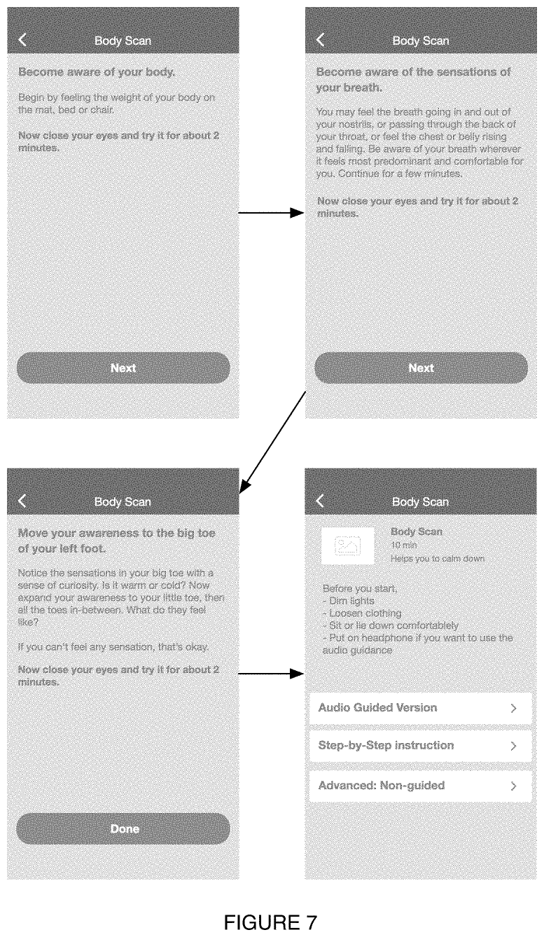

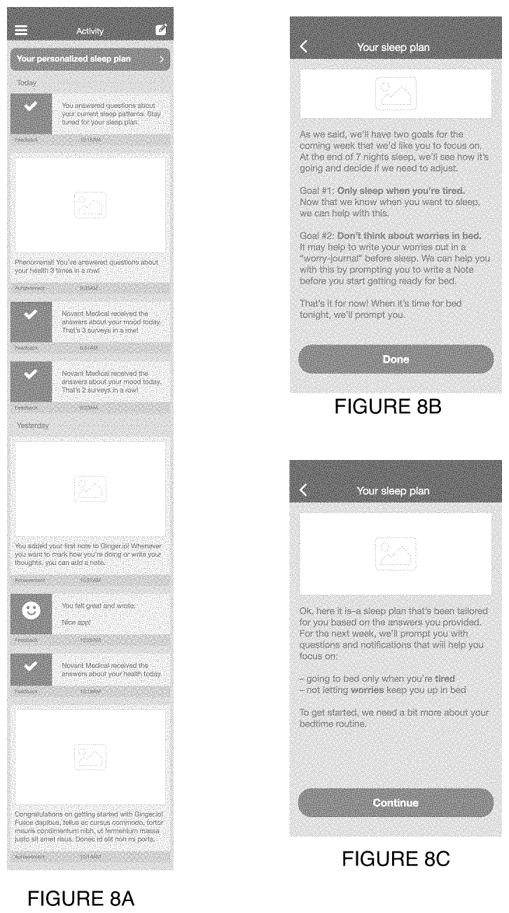

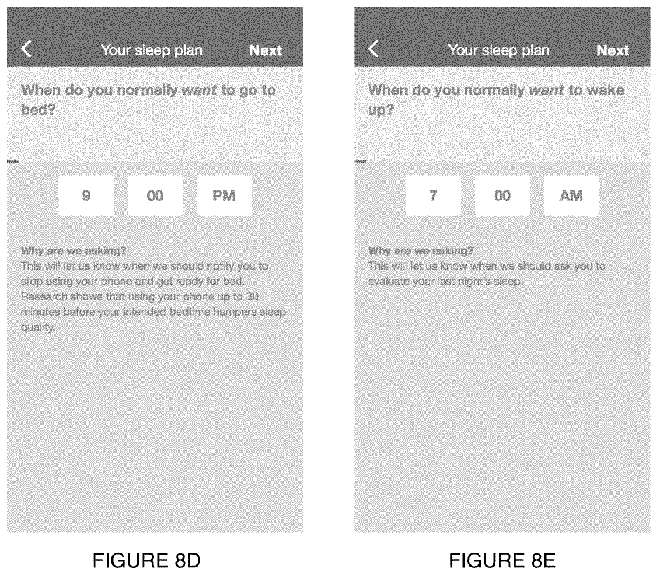

In another variation, Block S140 can include selecting a relaxation therapeutic intervention that allows the user to become more aware of his/her body, as shown in FIG. 7. In the relaxation therapeutic intervention, the user can be guided (e.g., visually, audibly), or unguided in becoming aware of his/her body in phases. In another example, the therapeutic intervention selected can include allowing the user to communicate with an entity that provides therapeutic communication to the user in improving his/her state. In another example related to sleep, as shown in FIGS. 8A-8E, the selected therapeutic intervention can provide the user with a summary of his/her progress in managing sleep behavior, and provide the user with a personalized sleep plan according to the user's desired sleep goals (e.g., wakeup times, bedtimes, etc.). However, the therapeutic intervention selected in Block S140 can additionally or alternatively include any other suitable therapeutic intervention.

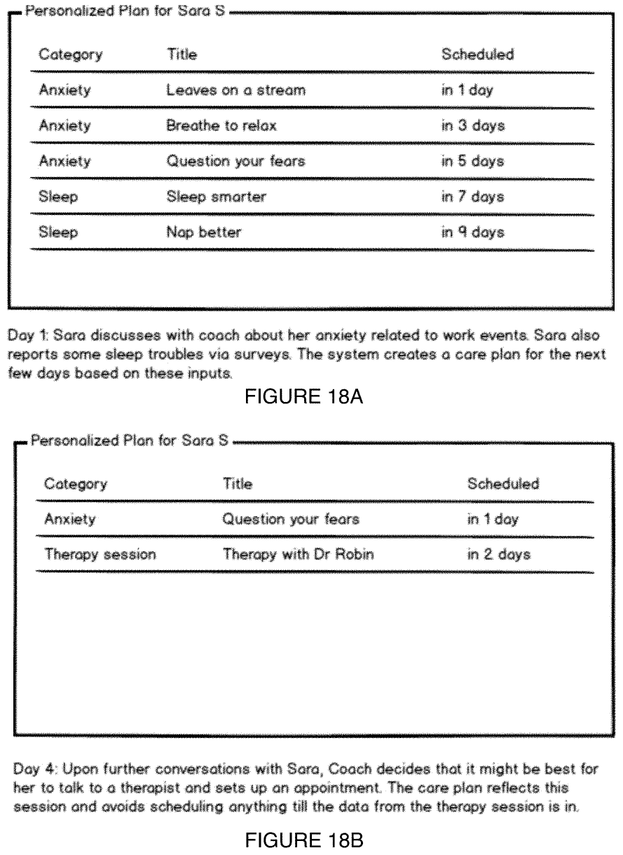

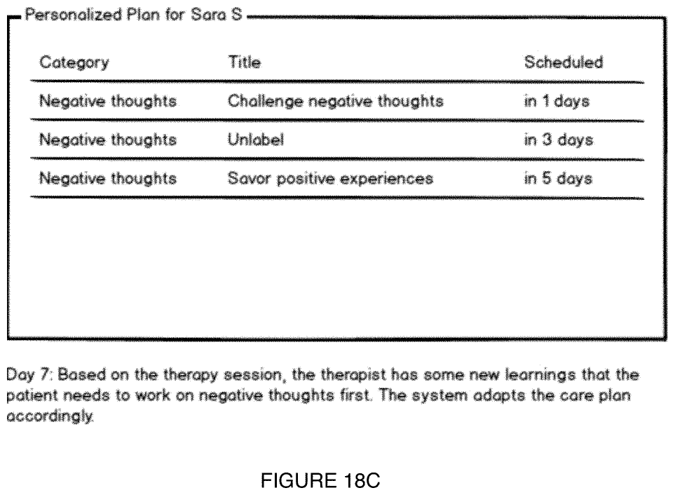

In another variation, Block S140 can include facilitating communication (e.g., text messaging, e-mail, phone calling, in-person communication, etc.) with one or more care providers. For example, as shown in FIG. 18B, Block S140 can include scheduling a care provider session for a patient. In a specific example, Block S140 can include facilitating a therapy session based on active and/or passive data indicating that patient issues would be better solved by the patient discussing past patient experiences with a therapist. In another specific example, Block S140 can include facilitating a psychiatrist session in response to stagnant patient improvement (e.g., automatically determined based on active data and/or passive data) with respect to symptoms and/or condition severity, and/or in response to indicators (e.g., care provider data, patient survey data, etc.) showing the a patient requires a medical diagnosis for their condition. Facilitating communication can be performed in response to manual triggers (e.g., a care provider selecting an option indicating that the care provider recommends direct communication with a care provider), automatic triggers (e.g., based on a therapeutic intervention model), and/or in any suitable manner). Therapeutic interventions including communications with a care provider can be automatically scheduled (e.g., added to a dynamic care plan), manually scheduled (e.g., by the care provider, where the dynamic care plan can adjust accordingly), and/or otherwise facilitated. Care providers can be matched to patients manually (e.g., by a human curator), automatically (e.g., through a matching model), and/or through any suitable means. For example, the method 100 can include: generating a patient therapy profile for the patient based on at least one of a log of use dataset, a mobility behavior supplementary dataset, and a patient interaction with a promoted therapeutic intervention; generating a comparison between the patient therapy profile and a care provider profile, and selecting a personalized care provider for the patient based on the comparison, wherein a personalized therapeutic intervention is digital communication with the personalized care provider. Patient profiles and/or care provider profiles can include information related to any one or more of: intervention specialty (e.g., a patient's preferred type of intervention category, a care provider's intervention category specialties), client type (e.g., adults, children, females, males), experience level, cost (e.g., a patient's preferred cost, a care provider's cost), location, and/or other suitable information. For example, generating a comparison between profiles can include generating the comparison between a preferred intervention category (e.g., from the patient therapy profile) and the intervention specialty (e.g., from the care provider profile). Additionally or alternatively, facilitating digital communication with a care provider can be performed in any manner analogous to embodiments, variations, and examples described in U.S. application Ser. No. 15/005,923, entitled “Method for Providing Therapy to an Individual” and filed on 25 Jan. 2016, which is herein incorporated in its entirety by this reference.

However, therapeutic intervention selection, types of therapeutic interventions, and therapeutic intervention-related concepts can include any matter analogous to embodiments, variations, and examples described in U.S. application Ser. No. 15/245,571, entitled “Method and System for Modeling Behavior and Heart Disease State” and filed on 24 Aug. 2016, which is herein incorporated in its entirety by this reference. Further, selecting a therapeutic intervention S140 can be performed in any suitable manner.

### 3.6.A Generating a Therapeutic Intervention Predictive Model

As shown in FIG. 1A, Block S142 recites: generating a therapeutic intervention predictive model derived from at least one of a log of use dataset, a supplementary dataset, a survey response dataset, and a behavioral dataset, which functions to provide a therapeutic intervention predictive model that can generate one or more outputs indicative of a preferred therapeutic intervention and/or a patient health state. Preferably, a therapeutic intervention predictive model outputs one or more values of a criticality parameter indicative of a critical state resolvable with one or more therapeutic interventions. In particular, the therapeutic intervention predictive model can determine a value of a criticality parameter in association with at least one time window (e.g., a time window within the time period, a time window outside of the time period based upon extrapolation) in predicting risk that the user is experiencing a critical symptomatic state, or will trend toward a critical symptomatic state at a future time point. Preferably, generation of the therapeutic intervention predictive model includes utilization of one or more machine learning techniques and training data (e.g., from the user, from a population of users), data mining, and/or statistical approaches to generate more accurate models pertaining to the user's disorder state (e.g., over time, with aggregation of more data). Additionally or alternatively, a therapeutic intervention predictive model can incorporate probabilistic properties, heuristic properties, deterministic properties, and/or any other suitable properties for generating a cardiovascular health metric.

Additionally or alternatively, Block S142 can include outputting one or more selections of therapeutic interventions (e.g., to be promoted in Block S160). Any number of therapeutic interventions can be selected, ranked, scored, and/or output in any suitable fashion. In an example, Block S142 can include selecting a subset of therapeutic interventions (e.g., a health tip prompting frequent communication with friends and a scheduled digital communication with a health coach) based on processing a therapeutic intervention predictive model with a log of use dataset (e.g., indicating that a patient likes to socialize). In another example, one or more therapeutic intervention models can output therapeutic intervention provision parameters (e.g., described in Block S160) indicating a recommended manner (e.g., when, how, at what device, etc.) for promoting one or more selected therapeutic interventions.

In generating the therapeutic intervention predictive model, Block S142 preferably uses input data including communication behavior data from the log of use, data from a supplemental dataset, data from the survey response dataset, and/or data from the behavioral dataset to provide a set of feature vectors corresponding to time points of the time period. Feature selection approaches can include one or more of: factor analysis approaches that implement statistical methods to describe variability among observed features in terms of unobserved factors, in order to determine which features explain a high percentage of variation in data; correlation feature selection (CFS) methods, consistency methods, relief methods, information gain methods, symmetrical uncertainty methods, and any other suitable methods of feature selection. In variations, feature selection approaches can be implemented for any passive data (e.g., communication data, mobility data), where a linking analysis of Block S142 is then used to determine associations between features of passive data and states of disorder determined from active data (e.g., survey response datasets). Analysis of the passive data in relation to the active data, with regard to feature selection and associations between passive and active data can, however, be performed in any other suitable manner.

In one variation, the feature vectors can include features related to aggregate communication behavior, interaction diversity, mobility behavior (e.g., mobility radius), a number of missed calls, and a duration of time spent in a certain location (e.g., at home). In examples, feature vectors can be generated based upon aggregation of phone, text message, email, social networking, and/or other user communication data for a particular period of time into one or more features for the user for the particular time period. Furthermore, a feature can be specific to a day, a week, a month, a day period (e.g., morning, afternoon, evening, night), a time block during a day (e.g., one hour), a specific communication action (e.g., a single phone call), a set of communication actions of the same type (e.g., a set of phone calls within a two-hour period, all communications within a period of time, etc.). Additionally, combinations of features can be used in a feature vector. For example, one feature can include a weighted composite of the frequency, duration (i.e., length), timing (i.e., start and/or termination), and contact diversity of all outgoing voice (e.g., phone call) communications and a frequency, length, and timing and/or response time to (i.e., time to accept) incoming voice communications within the first period of time through a phone call application executing on the user's mobile computing device. Feature vectors can additionally or alternatively include features based on analysis of voice communications, textual communications, mobile application activity usage, location data, and any other suitable data which can be based on variance, entropy, or other mathematical and probabilistic computations of basic data (e.g., a composite activity score, a composite socialization score, a work-life balance score, a quality-of-life score). However, the feature vectors can be determined in any other suitable manner.

In some variations, Block S142 can utilize statistics-based feature selection approaches to determine a subset of features from the log of use, an active dataset (e.g., survey dataset, care provider dataset, etc.), and/or the supplementary dataset that have a high predictive power and/or high accuracy in generating the value(s) of a criticality parameter as an output of the therapeutic intervention predictive model. Furthermore, the statistical approaches can be used to strategically reduce portions of data collected based upon redundancy and/or lack of utility of the data. Even further, the statistical approaches/feature selection approaches can be used to entirely omit collection of portions of the data (e.g., responses to specific surveys or portions of surveys can render responses to other portions of surveys or other surveys redundant), in order to streamline the data collection in Blocks S110, S120, and/or S130. In examples, the statistical approaches can implement one or more of: correlation-based feature selection (CFS), minimum redundancy maximum relevance (mRMR), Relief-F, symmetrical uncertainty, information gain, decision tree analysis (alternating decision tree analysis, best-first decision tree analysis, decision stump tree analysis, functional tree analysis, C4-5 decision tree analysis, repeated incremental pruning analysis, logistic alternating decision tree analysis, logistic model tree analysis, nearest neighbor generalized exemplar analysis, association analysis, divide-and-conquer analysis, random tree analysis, decision-regression tree analysis with reduced error pruning, ripple down rule analysis, classification and regression tree analysis) to reduce questions from provided surveys to a subset of effective questions, and other statistical methods and statistic fitting techniques to select a subset of features having high efficacy from the data collected in Blocks S110, S120, and/or S130. Additionally or alternatively, any assessment of redundancy or efficacy in a feature derived from data of Blocks S110, S120, and/or S130 can be used to provide a measure of confidence in a symptom criticality parameter produced by the therapeutic intervention predictive model from one or more input features.

In some embodiments, the therapeutic intervention predictive model generated in Block S142 can process a set of feature vectors according to methods described in relation to the therapeutic intervention predictive modeling engine described in U.S. application Ser. No. 13/969,339, entitled “Method for Modeling Behavior and Health Changes” and filed on 16 Aug. 2014, which is incorporated herein in its entirety by this reference; however, the therapeutic intervention predictive model can alternatively be generated in any other suitable manner. As such, in variations of the model(s), a set of feature vectors from the input data can be processed according to a machine learning technique (e.g., support vector machine with a training dataset) to generate the value(s) of the criticality parameter in association with a time point. In one example, the therapeutic intervention predictive model can incorporate historical data from the user (e.g., survey responses from a prior week, a history of passive data from the log of use, etc.), with more weight placed upon more recent data from Blocks S110-S130 in generation of the criticality parameter by the therapeutic intervention predictive model; however, the therapeutic intervention predictive model can be implemented in any other suitable manner.

However, generating a therapeutic intervention predictive model S142 can be performed in any suitable manner.

### 3.6.B Generating a Comparison Between a Dataset-Derived Component and a Threshold Condition.

As shown in FIG. 1A, Block S144 recites: generating a comparison between a dataset and a threshold condition. Block S144 functions to compare one or more threshold conditions against one or more datasets related to Block S110-S132, in order to select and/or promote a therapeutic intervention, generate and/or dynamically modify a care plan, and/or facilitate other suitable portions of the method 100. For example, selecting a therapeutic intervention (e.g., prompts for performing physical exercises) from a set of therapeutic interventions can be based on a patient health state (e.g. a lethargic health state for a diabetic patient) inferred from one or more datasets satisfying one or more threshold conditions (e.g., log of use data indicating high-sugar intake, and mobility behavior supplemental data indicating infrequent physical activity). In another example, automatically promoting a therapeutic intervention (e.g., prompting a patient to call a suicide hotline) can be in response to satisfaction of a threshold condition by a dataset (e.g., a digital communication indicating that a patient is going to commit suicide).

In variations, Block S144 can process data related to Blocks S110-S132, such that the patient health state determined in Block S146 can be derived from at least one of an active component (e.g., a component derived from the survey response dataset, a component derived from a care provider dataset such as text and/or audio conversations between a care provider and a patient), a passive component (e.g., a clinically-informed behavioral rule component determined by heuristics, digital communication behaviors indicated by a log of use dataset including text and/or audio conversations, mobility behaviors indicating by a supplemental dataset, other characteristics related to collected passive data), and a component derived from one or more therapeutic intervention predictive models generated in Block S142. In particular, consideration of the active component, a passive component, and/or a component derived from the therapeutic intervention predictive model can provide greater certainty in the state of the user determined in Block S144, which can significantly increase the efficacy of the therapeutic intervention(s) selected and provided to a user in Blocks S140 and S160, as shown in FIGS. 2A-2B.

Furthermore, an active component, a passive component, and/or a therapeutic intervention predictive model component can have an associated time frame that is identical or different to time frames of analysis of the other components. Additionally, analysis of each of the active component, the passive component, and the therapeutic intervention predictive model component can occur within one or more time frames that are different from the time frame of therapeutic intervention selection and provision in Blocks S140 and S160. In view of a population of users, consideration of the active component, the passive component, and the component derived from the therapeutic intervention predictive model facilitates prioritization of indications generated for different users, given, for instance, resource constraints in providing suitable therapeutic interventions for users in need.

Block S144 can optionally include generating a first comparison between a survey response dataset and a first threshold condition, which can include assigning a score to a survey response dataset for a patient (e.g., based upon one instance of survey response provision, based upon multiple instances of survey response provision), and comparing the score to the first threshold condition. In variations where the survey response dataset includes responses to survey questions (e.g., a repeat set of survey questions) at each of a set of time points, the first threshold condition can additionally or alternatively include a frequency threshold and/or a frequency-within-a-duration-of-time threshold, in relation to generation of an indication based upon an active component. Furthermore, threshold conditions can be defined in relation to a baseline for each user, based upon historical behavior of the user. As such, in variations, a comparison can indicate one or more of: a score greater than a given threshold; a score greater than a given threshold for a certain duration of time; a change in score greater than a given threshold; a change in score greater than a given threshold as derived from the user's historical score data; and any other suitable comparison. Furthermore, the comparison(s) can additionally or alternatively be generated based upon a percentile condition, a standard deviation (e.g., in score) condition, outlier detection analysis (e.g., of a score in relation to scores from the user), and/or any other suitable condition, based upon analysis of a user in isolation, based upon analysis of the user's recent behavior in isolation, based upon analysis of a population of users, and/or any other suitable grouping of users.

Additionally or alternatively, the comparison(s) generated in Block S144 can include identification or analysis of user progress through a condition (e.g., in relation to persistence of symptoms, in relation to worsening of symptoms, in relation to improvement of symptoms, etc.).

In examples, the comparison can facilitate identification of one or more of: a score for survey responses that surpasses a critical threshold score (e.g., a score above a critical value on a PHQ-9 survey); a change in survey score that surpasses a critical threshold; a set of scores for survey responses acquired at each of a set of time points within a duration of time, where a threshold proportion of the set of scores surpasses a critical threshold score (e.g., 2 of 3 surveys have scores above a critical threshold); a summation of scores for a set of scores for survey responses acquired at each of a set of time points that surpasses a critical threshold; a magnitude of difference in scores for survey responses acquired at different time points that surpasses a critical threshold (e.g., a PHQ-9 score >15, which is greater than a previous score); a combination of scores for different surveys that surpasses a critical threshold for each of the different surveys; and any other suitable condition for indication generation.

Block S144 can optionally include generating a second comparison between a second threshold condition and a log of use dataset, a supplementary dataset, and/or a behavioral dataset. The second comparison can include defining one or more passive behavior (e.g., related to lethargy, related to social isolation, related to physical isolation, related to evolution of the user's support network, related to time spent at work, related to weekly behavioral patterns, etc.) based upon historical behavior of a user within a duration of time (e.g., immediately prior 4-6 weeks of the user's life). Then, the features of or evolution in the passive heuristic(s) for the user can be compared to the second threshold condition. In variations where the passive behavior for the user are monitored for a duration of time, the second threshold condition can additionally or alternatively include a frequency threshold and/or a frequency-within-a-duration-of-time threshold, in relation to generation of an indication based upon a passive component. In examples, the comparison can facilitate identification of one or more of: a period of lethargy exhibited as a persistent reduction in mobility (e.g., little motion over a period of 3 consecutive days); a period of social isolation exhibited as persistence in unreturned communications (e.g., a period of 3 days of unreturned phone calls, a period of 3 days of unreturned text-based communications, etc.); a period of physical isolation exhibited as persistence in staying in a location (e.g., staying primarily at the same location for a period of 3 or more days); a reduction in the user's support network exhibited as communicating with fewer people than typical for the user; a combination of multiple passive behavior that satisfy a threshold condition (e.g., two passive behavior that meet a threshold within 3 days); and any other suitable condition for indication generation.

Block S144 can optionally include generating a third comparison between the output of the therapeutic intervention predictive model and a third threshold condition. The third comparison can include identification of a classification (e.g., a learned, complex, non-intuitive, and/or behavioral association exhibited by the user), and comparing the classification to a threshold condition. In variations, a single feature and/or combinations of features derived from the log of use, the survey response dataset, and the behavioral dataset (e.g., with weighting among factors) can be compared to one or more threshold conditions, in identifying if an indication based upon the therapeutic intervention predictive model of Block S142 should be generated. In variations and examples, the third comparison can be generated as described in U.S. application Ser. No. 13/969,339, entitled “Method for Modeling behavior and Health Changes” and filed on 16 Aug. 2014.

As such, in one example of Block S144, accounting for an active component, a passive component, and a therapeutic intervention predictive model component, an indication can be based upon: scoring of a biweekly survey, a first passive component generated from a first 3-day window of time, a second passive component generated from a second window of time overlapping with the first 3-day window of time, and a therapeutic intervention predictive model component for a time window of 14 days (e.g., overlapping with the period of the biweekly survey), where the therapeutic intervention predictive model component implements an aggregated learning approach based upon multiple individual models (e.g., each assessing different parameters and/or different time periods of user behavior).

However, generating a comparison between a dataset and a threshold condition S144 can be performed in any suitable manner.

### 3.6.C Determining a Patient Health State.

As shown in FIG. 3A, Block S146 recites: determining a health state of the patient during a time period, which functions to identify if the user is experiencing an adverse health state that could be improved by a therapeutic intervention.

In Block S146, determining a patient health state can be based on processing with a therapeutic intervention predictive model (e.g., described in Block S142), comparisons with thresholds (e.g., described in Block S144), comparisons with reference profiles (e.g., associated with a patient health state), and/or other suitable approaches.

In Block S146, the state of the user is preferably determined from the outputs of Blocks S110-S144 in near-real time or substantially in real time, such that an identified state of the user can be responded to with an appropriate therapeutic intervention in near-real time or substantially in real time, if needed. Additionally or alternatively, the state of the user can be determined in non-real time (e.g., in post-processing) or in any other suitable manner. For instance, in some variations, an therapeutic intervention can be provided to the user at any suitable point in relation to a timespan of a health condition-related episode (e.g., at an earlier stage of an episode, at a later stage of an episode), or when a user is in a suitable state or environment to receive an therapeutic intervention (e.g., when the user is at home).

In Block S146, the time point can be a future time point, such that the state of the user determined in Block S146 is a predicted state that the user is expected to trend toward, if no therapeutic intervention is provided to the user (as in Blocks S140 and S160). The time point can additionally or alternatively be a substantially current time point or a time point in the past, such that the state determined in Block S146 is a substantially present state of the user or a past state of the user. As such, Block S146 can additionally or alternatively include determining a trend in the state of the user over a set of time points, where the set of time points can be regularly spaced (e.g., at a set frequency) or irregularly spaced, and include one or more of: past time points, a present time point, and future time points.

For Block S146, in relation to the first comparison, the second comparison, and/or the third comparison, the state of the user output by Block S146 can be described based upon a combination of information from at least one of Blocks S110-S142. As such, the determined state can be based upon combinations of active data (e.g., survey data, care provider data, etc.), passive data (e.g., behavioral data), and therapeutic intervention predictive models.

In more detail, in Block S146, the determined states(s) of the user can have one or more hierarchies of descriptiveness. Furthermore, the determined state(s) of the user can be qualitative (e.g., in providing qualitative descriptions of a user state) and/or quantitative (e.g., in providing a value of a metric). In one variation, a first hierarchy level can qualitatively describe a general state of the user (e.g., great, fine, at-risk, critical, etc.), a second hierarchy level can further describe the state of the user in relation to phases of a health condition (e.g., a psychological disorder, a condition of depression, a pain-related condition, a sleep-related condition, a cardiovascular disease related condition, etc.), and a third hierarchy level can further provide quantitative values of a metric that characterizes severity of the health condition of the user. In an example of this variation, a state of the user can thus output a state of the user as the following: at-risk of entering a critical state of psychosis, and a severity level of 7 (on a 1-10 scale), as determined from an average Brief Psychiatric Rating Scale score of 4 and a reduced level of mobility. However, variations of Block S146 can output a state of the user in any other suitable manner, with any other suitable parameters for characterizing the state of the user. Furthermore, Block S146 can include holistically describing a health state of the user in relation to multiple health conditions.

In a variation, Block S146 can include mapping one or more patient health states to one or more therapeutic interventions (e.g., for facilitating selection of therapeutic interventions based on determined patient health states). For example, the method 100 can include mapping a health state to a first intervention category characterizing a first therapeutic intervention, based on an association between the health state and the first intervention category (e.g., where the first intervention category is from a set of intervention categories comprising at least one of: a psychiatric management category, pharmacotherapeutic category, and a behavioral therapy category). In this example, the method 100 can further include determining a change in health state of the patient from the first therapeutic intervention; in response to the change in health state below a health state threshold, dynamically modifying the dynamic care plan to include a second therapeutic intervention characterized by a second invention category from the set of intervention categories, wherein the first intervention category is different form the second intervention category; and automatically promoting the therapeutic intervention according to the modified dynamic care plan. Mapping patient health states to therapeutic interventions can be performed manually (e.g., predetermined with human intervention), automatically (e.g., based on known data indicating therapeutic interventions types likely to improve a given patient health state; based on collected data evaluating efficacy of intervention types for given patient profiles, etc.), and/or in any suitable manner. Generated mappings can be updated based on data related to Blocks S110-S144 and/or other suitable data.

However, determining a patient health state S146 can be performed in any suitable manner.

### 3.7 Generating a Dynamic Care Plan.

As shown in FIGS. 1A-1B, and 2A, Block S150 recites: generating one or more dynamic care plan for the patient. Block S150 functions to create an adaptable plan for providing psychological and/or physiological health in a personalized manner.

As shown in FIGS. 18A-18C, the dynamic care plan preferably specifies the manner in which one or more therapeutic interventions (e.g., selected in Block S140) are administered for a patient. As such, a dynamic care plan preferably includes at least one therapeutic intervention, but can alternatively omit therapeutic interventions (e.g., a dynamic care plan focused on providing patient health information to care providers; a dynamic care plan specifying optimal times to provide an intervention, without including the interventions themselves, etc.). For example, generating a dynamic care plan can include selecting a first and a second therapeutic intervention (e.g., in Block S140). In another example, Block S150 can include generating a dynamic care plan modifiable over a second time period subsequent the first time period, the dynamic care plan including a therapeutic intervention. In another example, generating a digital care plan can include selecting a second therapeutic intervention from the set of therapeutic interventions, based on processing at least one of a log of use dataset and a mobility behavior supplementary dataset with the therapeutic intervention predictive model, where the second therapeutic intervention is distinct from a first therapeutic intervention and a personalized therapeutic intervention (e.g., selected for dynamic modification of the dynamic care plan), wherein the first therapeutic intervention is from a behavioral therapy intervention category, wherein the second therapeutic intervention is from a biosignal-related intervention category, and the method further including after promoting the first therapeutic intervention at the mobile computing device, promoting the second therapeutic intervention at a biosignal detector coupled to the patient, according to the digital care plan. In this example, the first therapeutic intervention can be a cognitive behavioral therapy exercise, the second therapeutic intervention can be EEG biosignal collection, and the method 100 can further include: substantially concurrently with promoting the cognitive behavioral therapy exercise at the mobile computing device, controlling an EEG biosignal detector, coupled to the patient, to perform the EEG biosignal collection according to the dynamic care plan.

Further, in relation to Block S150, generating the dynamic care plan preferably includes determining therapeutic intervention provision parameters for one or more therapeutic interventions. Therapeutic intervention provision parameters can include any one or more of: temporal parameters (e.g., when to schedule the promotion of a therapeutic intervention, how frequently to promote the therapeutic intervention, etc.), venue parameters (e.g., at which mobile computing device to promote the therapeutic intervention, to which individuals should the therapeutic intervention be promoted, etc.), threshold parameters (e.g., conditions triggering promotion of a therapeutic intervention, etc.), and/or any suitable parameters. For example, Block S150 can include scheduling, through a telecommunications API, administration of the therapeutic intervention (e.g., a health-related notification pushed to the phone) for a time window (e.g., a time window during the day when the patient has a high amount of digital communication through the mobile computing device) based on a temporal therapeutic intervention temporal parameter. In another example, the method 100 can include generating a dynamic care plan specifying a scheduled time window in which to promote a therapeutic intervention; dynamically modifying the scheduled time window based on at least one of a second log of use dataset and a second mobility behavior supplementary dataset (e.g., collected after generation of the dynamic care plan), each dataset corresponding to the second time period (e.g., after a first time period prior to generating the dynamic care plan), wherein automatically promoting the first therapeutic intervention comprises automatically promoting the first therapeutic intervention during the modified scheduled time window. In another example, Block S150 can include establishing a threshold parameter of promoting a therapeutic intervention (e.g., a nightly interactive mood survey) when the patient is in bed (e.g., a time period in which log of use data for the patient indicate a low level of digital communication from the patient); and identifying that the patient is in bed based on a supplementary dataset (e.g., accelerometer data indicating a low degree of physical activity; location data indicating that the patient is in their bedroom; light sensor data indicating the lack of light; time of day, etc.) collected at a mobile computing device (e.g., a sensor of the mobile computing device).

Additionally or alternatively, for Block S150, the dynamic care plan can include patient-related information (e.g., patient state, recommended therapeutic interventions for the patient, digital communication behavior information, mobility-related information, etc.) guiding one or more care providers in providing health care to the patient, but the dynamic care plan can be otherwise defined.

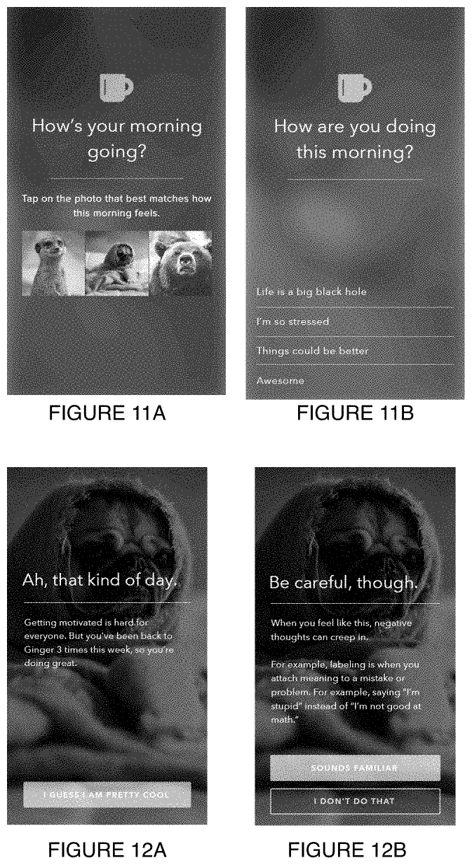

The dynamic care plan is preferably generated based on at least one of log of use data, supplemental data, active data (e.g., survey data, care provider data, etc.), and one or more outputs of a therapeutic intervention predictive model, but can additionally or alternatively be generated from any suitable data. In an example, Block S150 can include generating the dynamic care plan based on active data collected from care providers (e.g., doctors appointments, therapy sessions), coaches, family/friends (e.g., who are also on the platform etc.), other users on the platform (e.g., support groups, message boards etc. facilitated through the platform), and/or other individuals. In another example, Block S150 can include generating a dynamic care plan, including scheduling promotion of a therapeutic intervention based on location data extracted from a mobility behavior supplementary dataset (e.g., scheduling skill-building exercises for when the patient is located in a non-professional setting). In another example, the method 100 can include: initiating, at the mobile computing device, a digital care plan calibration survey (e.g., a set of image options representing different moods as shown in FIG. 11A); receiving, at the mobile computing device, a patient response to the digital care plan calibration survey (e.g., a selection of an image from the options; and generating a dynamic care plan, including selecting a therapeutic intervention based on processing a therapeutic intervention predictive model with the patient response (e.g., selecting a therapeutic intervention focused on improving stress in response to a patient selecting an image representing anxiety). In another example, the method 100 can include: automatically initiating a digital communication between a patient at the mobile computing device and a care provider at a care provider device; receiving, at a digital interface provided to the care provider at the care provider device, a care provider dataset including patient information derived from the digital communication, wherein selecting the first therapeutic intervention is based on processing a therapeutic intervention predictive model with the care provider dataset.

In another example of Block S150, therapeutic interventions and/or therapeutic intervention provision parameters of a dynamic care plan can be influenced from user preferences including at least one of preferred types of therapeutic interventions and/or therapeutic intervention categories, preferred therapeutic intervention provision parameters (e.g., a selection of personal contacts to contact for therapeutic interventions involving other individuals, etc.). User preferences can be selected manually (e.g., by a patient based on options provided at an application executing on a patient mobile computing device, etc.), automatically (e.g., inferred from log of use data, supplemental data, survey data, etc.), and/or through any other means.

In a variation, Block S150 can include generating a dynamic care plan based on manual input by a care provider or other entity. For example, generating a dynamic care plan can be based on care provider data and/or manual input by the care provider based on their interactions with the patient, based on their review of collected active and/or passive data (e.g., aggregated in a report for the care provider to evaluate), and/or based on any suitable criteria. In another example, Block S150 can include transmitting generated dynamic care plans (e.g., automatically and/or manually curated) to a care provider for approval. In this example, Block S150 can include receiving manual input by the care provider that can be used in updating the dynamic care plan (e.g., to delete a therapeutic intervention, to add a therapeutic intervention, to modify a therapeutic intervention provision parameter, etc.)

In a variation, Block S150 can include venue parameters specifying a set of patient devices through which to promote one or more therapeutic interventions. In this variation, the venue parameters can include a priority ranking of patient devices, specifying high ranked patient devices at which to promote a therapeutic intervention before attempting to promote the therapeutic intervention at lower ranked patient devices. Establishing a hierarchy of patient devices can increase the probability of a patient receiving and/or responding to a delivered therapeutic intervention. In an example, generating a dynamic care plan can include specifying a secondary device at which to promote the therapeutic therapy in response to a failed attempt to establish a wireless communicable link with a primary device (e.g., where the primary and secondary devices are a mobile computing device and a cardiovascular device, respectively, and where automatically establishing the wireless communicable link with the cardiovascular device is in response to failing to establish a wireless communicable link with the mobile computing device).

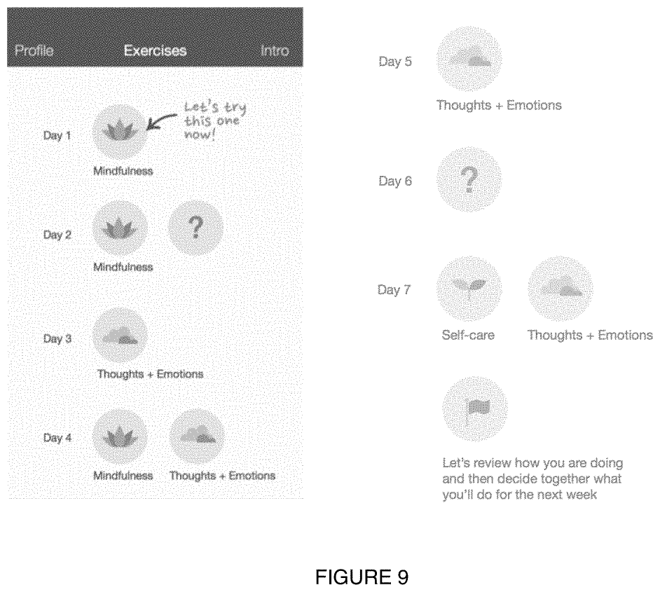

In a variation, as shown in FIG. 9, Block S150 can include presenting the dynamic care plan to a patient (e.g., for tracking progress), family/friends (e.g., for monitoring patient progress), care providers (e.g., for tailoring their healthcare approaches based on the presented dynamic care plan), and/or any other suitable entity.

However, generating a dynamic care plan S150 can be performed in any suitable manner.

### 3.8 Promoting a Therapeutic Intervention.

As shown in FIGS. 1A-1B, 2A-2B, and 3A, Block S160 recites: promoting a therapeutic intervention to the patient according to a dynamic care plan, which functions to provide the therapeutic intervention to the user when the user is amenable to responding to and/or receiving the therapeutic intervention. The therapeutic intervention can be provided at a single temporal indicator (e.g., time point, time window, time period, etc.), or can additionally or alternatively be provided according to a schedule or a regimen (e.g., based on temporal parameters of a dynamic care plan), an example of which is shown in FIG. 9, such that portions of the therapeutic intervention(s) are delivered to the user at a set of temporal indicators (e.g., different days). Different therapeutic interventions can be delivered, and/or the same therapeutic intervention can be repeatedly delivered (e.g., at different temporal indicators). Promotion of the therapeutic intervention can be facilitated through one or more of: mobile computing device (e.g., an application executing on the mobile computing device, a tablet, personal computer, cardiovascular device, biosignal detector head-mounted wearable computing device, wrist-mounted wearable computing device, etc.), a web application accessible through an internet browser, an entity (e.g., caretaker, spouse, healthcare provider, relative, acquaintance, etc.) trained to provide the therapeutic intervention, and in any other suitable manner. For example, Block S160 can include: automatically establishing a wireless communicable link with a cardiovascular device associated with the patient; and delivering, through the wireless communicable link, the therapeutic intervention to the patient at the cardiovascular device (e.g., presenting a health tip, prompting a skill-based exercise, transmitting a request to the cardiovascular device to monitor a cardiovascular parameter, to provide a cardiovascular therapy, etc.). In another example, Block S160 can include controlling a patient device to promote one or more therapeutic interventions, such as controlling operation of a smart lighting system (e.g., Phillips Hue™, LIFX™, etc.) to emit light at a lighting setting (e.g., a warmer, softer color temperature) configured to improve a patient health state (e.g. an anxious mental state). In a variation, Block S160 includes promoting a therapeutic intervention at multiple patient devices (e.g., presenting a health tip at a patient's smart phone and a patient's smart watch).

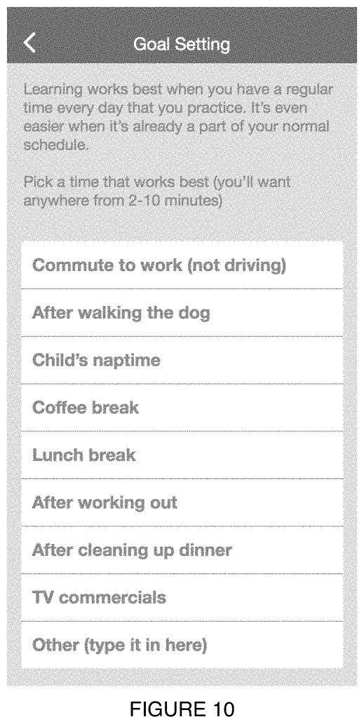

In Block S160, a therapeutic intervention can be delivered based on the dynamic care plan, delivered immediately to the user (e.g., in response to selection of the therapeutic intervention in Block S140), delivered upon selection by the user, delivered proximal to a determining a health state of the user (e.g., anticipated state, current state, etc.) or behavioral state (e.g., lethargy, isolation, restlessness, etc.) known/presumed to impact the user's condition (e.g., based upon severity of the health state of the user determined in Block S146), and/or at any suitable time. Additionally or alternatively, the therapeutic intervention can be delayed until a time at which the user is anticipated to be most receptive to the therapeutic intervention. In one example, as shown in FIG. 10, the user can provide the system with an indication of when he or she is most likely to be receptive for a therapeutic intervention/reminder, such that the therapeutic intervention is provided during time blocks and/or within locations at which the user is most receptive, according to the indication. In another example, Block S160 can include determining receptiveness of the user (e.g., by calculating a receptiveness metric), based upon processing of contextual information (e.g., sensor and communication data received similar to the methods described in Block S110) for the user. For instance, if sensor data indicates that the user is immobile and/or asleep (e.g., based upon a lack of motion from accelerometer data), provision of a therapeutic intervention to the user can be delayed. In another example, if mobile device module activation data (e.g., phone usage data, app usage data) indicates that the user is unlikely to be using the mobile device, provision of a therapeutic intervention to the user can be delayed. In another example, if sensor data indicates that the user is in an environment (e.g., movie theater, board meeting, etc.) where he/she would be less receptive to an therapeutic intervention, provision of the therapeutic intervention to the user can be delayed. Additionally or alternatively, if a calendar of the user indicates that the user has a weekly commitment, provision of the therapeutic intervention to the user can be performed outside of the time block associated with the weekly commitment. However, the therapeutic intervention can be provided to the user in any other suitable manner, for any other suitable purpose (e.g., increasing or maintaining user engagement).

However, promoting a therapeutic intervention S160 can be performed in any suitable manner.

### 3.9 Dynamically Modifying a Dynamic Care Plan.

As shown in FIGS. 1A-1B and 2A, the method 100 can additionally or alternatively include Block S170, which recites dynamically modifying a dynamic care plan for a patient, thereby generating a personalized care plan. Block S170 functions to dynamically adjust a care plan (e.g., generated in Block S150) to better suit an individual's health state and/or behavior.

For Block S170, modifying a dynamic care plan preferably includes modifying types and/or categories of therapeutic interventions to promote, therapeutic intervention provision parameters, and/or any suitable parameters related to therapeutic interventions. Additionally or alternatively modifying a dynamic care plan can include modifying patient-related information included in a dynamic care plan, but any suitable data can be adjusted.

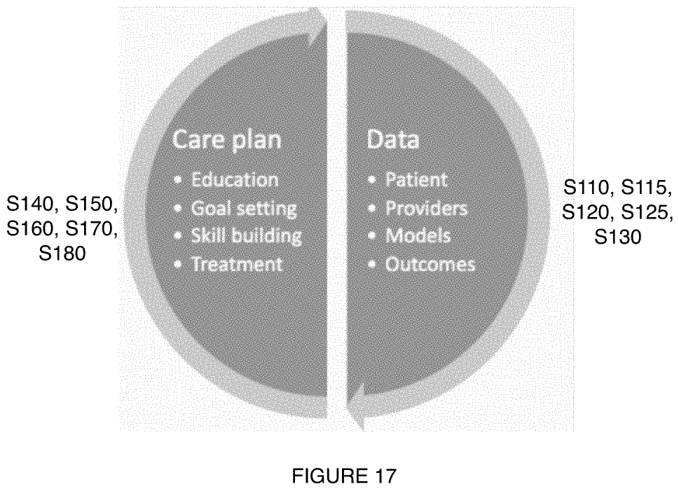

As shown in FIGS. 2A and 17, in relation to Block S170, dynamically modifying the dynamic care plan is preferably based on processing datasets (e.g., passive data, active data, processed data, etc.) received and/or collected subsequent to generation of an initial dynamic care plan (e.g., in Block S150) and/or promotion of one or more therapeutic interventions (e.g., in Block S160). For example, the method 100 can include: receiving a second log of use dataset associated with the patient digital communication behavior at a mobile computing device, wherein the second log of use dataset corresponds to a second time period subsequent a first time period (e.g., a time period when a first log of use dataset and supplementary dataset was received prior to generation of the dynamic care plan); and receiving a second mobility behavior supplementary dataset associated with a mobility-related sensor of the mobile computing device, wherein the second mobility behavior supplementary dataset corresponds to the second time period, wherein dynamically modifying the digital care plan is based on a received patient interaction (e.g., with the therapeutic intervention) and at least one of the second log of use dataset and the second mobility behavior supplementary dataset.

For Block S170, dynamically modifying one or more dynamic care plans can include processing datasets with one or more of: therapeutic intervention predictive models (e.g., described in Block S142), threshold conditions (e.g., described in Block S144), reference profiles, user preferences, and/or other suitable approaches. For example, the method 100 can include receiving patient interactions with one or more therapeutic interventions (e.g., the initial therapeutic interventions promoted in a dynamic care plan) at a patient device (e.g., patient mobile computing device); extracting a patient behavior from the patient interactions (e.g., inferring a patient preference for educational interventions); and dynamically modifying the dynamic care plan based on the extracted patient behavior (e.g., including more types of educational interventions). In another example, the method 100 can include dynamically modifying the digital care plan based on a received patient interaction with a therapeutic intervention, thereby generating a modified digital care plan including a personalized therapeutic intervention for the patient, wherein the personalized therapeutic intervention is distinct from the therapeutic intervention. In another example, the method 100 can include generating a dynamic care plan defining a set of skill paths (e.g., a sleep improvement skill path, a mindfulness skill path, etc.); identifying a satisfaction of goals for a first skill path (e.g., the sleep improvement skill path) based on analyzing a log of use dataset (e.g., indicating less anxiety and increased energy) and a mobility behavior supplemental dataset (e.g., indicating increased physical activity); and dynamically adjusting the dynamic care plan to transition into therapeutic intervention configured to satisfy goals for a second skill path (e.g., the mindfulness skill path). In another example, the method 100 can include: controlling the mobile computing device to emit a first audio therapy in response to receiving the patient interaction with the first therapeutic intervention, where the patient interaction is a user selection of the audio therapy from a set of therapies, and where dynamically modifying the digital care plan comprises selecting a second audio therapy for the patient based on the user selection of the first audio therapy, where the second audio therapy is the personalized therapeutic intervention.

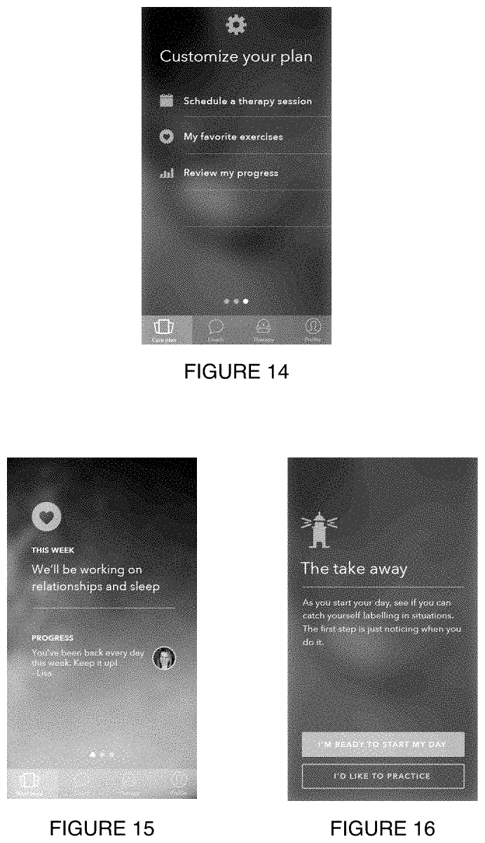

In a variation, as shown in FIG. 14, Block S170 can include dynamically modifying a dynamic care plan based on an active dataset (e.g., collected from a patient, care provider, friend, family, etc.). For example, the method 100 can include promoting, at a mobile computing device, a digital survey (e.g., asking how the patient feels after the therapeutic intervention) substantially concurrently with promoting a first therapeutic intervention; receiving, at the mobile computing device, a digital patient response to the digital survey; and generating an evaluation of improvement in the patient to the first therapeutic intervention, based on at least one of the digital patient response to the digital survey and the patient interaction with the first therapeutic intervention, where dynamically modifying the digital care plan comprises selecting the personalized therapeutic intervention for the patient based on the evaluation of improvement. In this example, the method 100 can optionally include: presenting, to a care provider at a web interface, patient information derived from the evaluation of improvement; and prompting the care provider at the web interface for care provider input on the digital care plan, where prompting the care provider is substantially concurrent with presenting the patient information the care provider, where selecting the personalized therapeutic intervention is based on the care provider input.

In relation to Block S170, dynamically modifying a dynamic care plan can be performed at predetermined time intervals (e.g., applying a therapeutic intervention predictive model every day, every week, every month, etc.), automatically determined temporal indicators (e.g., based on dynamic care plan temporal parameters determined when the initial dynamic care plan was generated), dynamically determined temporal indicators (e.g., based on satisfaction of a collected data amount threshold, based on digital communication inactivity of the patient, based on identification of a particular patient health state such as deteriorating health state, etc.), in response to a manual request (e.g., by a patient, care provider, etc.). For example, Block S170 can include receiving care provider input (e.g., in the form of care provider data), and modifying the dynamic care plan based on the care provider input. Block S170 can include transmitting the dynamic care plan Health coaches can review a dynamic care plan, a generated dynamic care plan (e.g., to delete a therapeutic intervention, to add a thereapeutic intervention, to modify a therapeutic intervention provision parameter, etc.) based on their interactions with the patient, based on their review of collected active and/or passive data (e.g., aggregated in a report for the care provider to evaluate), and/or based on any suitable criteria. In another example, Block S150 can include transmitting dynamic care plans (e.g., automatically and/or manually curated) to a care provider for approval. In this example, Block S150 can include receiving manual input by the care provider that can be included in updating the dynamic care plan.

For example, Block S170 can include selecting a personalized therapeutic intervention for modifying a dynamic care plan in response to patient completion of a default therapeutic intervention. In a variation, Block S170 can include dynamically modifying a set of dynamic care plans for a set of patients. Improved outcomes for multiple patients can be obtained from inferences regarding a single patient's progress with respect to therapeutic interventions promoted to the single patient. For example, the method 100 can include classifying a first patient into a subgroup of patients (e.g., high anxiety patients); promoting therapeutic interventions (e.g., cognitive behavioral therapy educational games); to the first patient; generating an evaluation of improvement in the first patient (e.g., in Block S180); and dynamically updating dynamic care plans for the subgroup of patients based on the therapeutic interventions and the evaluation of improvement (e.g., increasing the frequency of promoting cognitive behavioral therapy educational games to the subgroup of patients based on high efficacy for the first patient). In another example, the method 100 can include generating an evaluation of improvement in a first patient to a first therapeutic intervention; updating the therapeutic intervention predictive model with the log of use data, the mobility behavior supplementary dataset (e.g., the log of use data and the mobility behavior supplementary data leading to selection of the first therapeutic intervention), and the evaluation of improvement; selecting a second therapeutic intervention from the set of therapeutic interventions, based on processing with the updated therapeutic intervention predictive model; and generating a second dynamic care plan for a second patient, the second dynamic care plan including the second therapeutic intervention.

In a variation, as shown in FIG. 1A, Block S170 can include presenting modified dynamic care plans (e.g., by way of a mobile computing device notification indicating that an adjustment to the care plan was made) to a suitable individual (e.g., patient, care provider, etc.).

However, dynamically modifying one or more dynamic care plans S170 can be performed in any suitable manner.

### 3.10 Evaluating Patient Improvement.

As shown in FIGS. 1A-1B and 17, the method 100 can additionally or alternatively include Block S180, which recites generation an evaluation of patient improvement to one or more promoted therapeutic interventions. Block S180 functions to assess how a patient is responding to one or more therapeutic interventions and/or care plans, in order to provide actionable data for selecting more appropriate interventions and/or dynamically modifying dynamic care plans. Additionally or alternatively, generating an evaluation can function to provide a tracking measure for presenting progress to a patient, care providers (e.g., for directing health care provision, for facilitate health coaching). Additionally or alternatively, generating an evaluation can function to prompt further research down a line of interventions (e.g., directing research & development, clinical validation of newly formulated therapeutic interventions, etc.).

Regarding Block S180, generating an evaluation preferably includes calculating a patient improvement metric, which can be in one or more forms including: numerical (e.g., patient health state from 1-10 tracked over time, percentage increase in patient health state, etc.), verbal (e.g., describing mental health, mood, etc.), and/or in any suitable form.

For Block S180, generating a patient improvement evaluation is preferably based on passive data (e.g., log of use data, supplemental data, etc.). For example, the method 100 can include deriving a patient improvement metric for a therapeutic intervention based on a correlation between patient mood and log of use data (e.g., a negative correlation between depression and amount of digital communication); and updating the dynamic care plan based on the patient improvement metric (e.g., increasing the frequency of health tips prompting frequent communication with loved ones in response to an increased amount of patient digital communication from presenting such a health tip).

Additionally or alternatively, generating a patient improvement evaluation can be based on active data. In a variation, Block S180 can include administering (e.g., automatically administering, automatically administering based on a trained model, etc.) digital surveys before, during, and/or after promotion of a therapeutic intervention in order to analyze patient improvement from the therapeutic intervention. In examples, the surveys can be informal (e.g., “choose a face that best matches your current emotions”, asking the user to select an image from a set of images representing different emotions, etc.), formal (e.g., PHQ9, GAD7, etc.), and/or include any suitable information.

In relation to Block S180, generating one or more evaluations can be performed continuously, in response to a condition (e.g., promotion of a therapeutic intervention, updating of a dynamic care plan, etc.), at predetermined time intervals (e.g., as part of a daily patient assessment), determined with one or more trained models, and/or at any suitable time.

However, generating a patient improvement evaluation S180 can be performed in any suitable manner.

The method 100 can, however, include any other suitable blocks or steps configured to provide health therapeutic interventions to a user. Furthermore, as a person skilled in the art will recognize from the previous detailed description and from the figures and claims, modifications and changes can be made to the method 100 without departing from the scope of the method 100.

### 4. System.

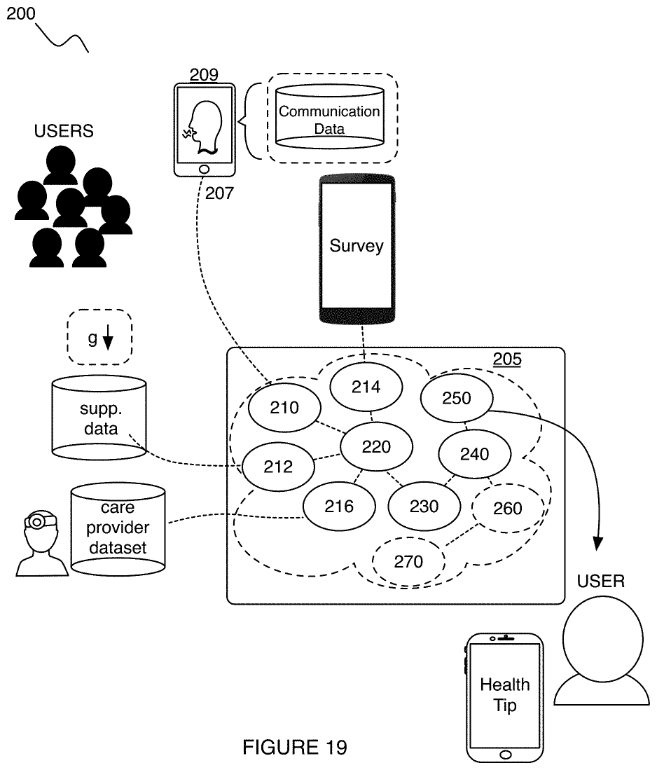

As shown in FIG. 19, a system 200 for providing health therapeutic interventions to a user includes: a processing system 205 including: interfaces 207 with data collection applications executing on mobile computing devices 209 of a population of users; a first module 210 configured to receive a log of use dataset; a second module configured to receive a supplementary dataset 212; a third module configured to administer and/or receive a survey dataset 214; a fourth module configured to receive a care provider dataset 216; a fifth module configured to process a dataset (e.g., generate a behavioral dataset) 220; a sixth module configured to select a therapeutic intervention from a set of therapeutic interventions 230; a seventh module configured to generate a dynamic care plan 240; an eighth module configured to promote a therapeutic intervention 250. The system 200 can additionally or alternatively include: a ninth module configured to dynamically modify a dynamic care plan 260; and/or a tenth module configured to evaluate patient improvement 270.

The system 200 functions to perform at least a portion of the method 100 described in Section 1 above, but can additionally or alternatively be configured to perform any other suitable method for providing health therapeutic interventions to a user. The system 200 is preferably configured to facilitate reception and processing of a combination of active data (e.g., inputs provided by individuals, post-communication survey responses) and passive data (e.g., unobtrusively collected communication behavior data, mobility data, etc.), but can additionally or alternatively be configured to receive and/or process any other suitable type of data. As such, the processing system 205 can be implemented on one or more computing systems including one or more of: a cloud-based computing system (e.g., Amazon EC3), a mainframe computing system, a grid-computing system, and any other suitable computing system. Furthermore, reception of data by the processing system 205 can occur over a wired connection and/or wirelessly (e.g., over the Internet, directly from a natively application executing on an electronic device of the individual, indirectly from a remote database receiving data from a device of the individual, etc.).

The method 100 can therefore be implemented using mobile computing devices, which can include any one or more of a smartphone, a digital music player, a tablet computer, a cardiovascular device (e.g., a cardiovascular monitoring device, a cardiovascular therapy device, etc.), a biosignal detector (e.g., an EEG headset, a ECG monitor, a heart rate monitor, etc.), a wrist-borne mobile computing device, a head-mounted mobile computing device, etc.) executing a native application, where the mobile computing devices receive inputs derived from behaviors of users and transmits data derived from the inputs to the processing system, and where the processing system generates and provides indications to entities based upon processing of the data. At least one element of the system preferably includes or is coupled to a display such that the method 100 can display information to an entity (e.g., a nurse, anesthesiologist, physician, caretaker, relative, acquaintance, etc.) and/or a user through the display (e.g., of a mobile computing device), in order to drive therapeutic interventions (e.g., in-application therapeutic interventions, therapies, health advice, etc.). Additionally or alternatively, the entity can be a computing system platform (e.g., processing system providing outputs to a dashboard), or any other suitable entity. However, the method 100 can alternatively be implemented using any other suitable system configured to process communication and/or other behavior of users, in aggregation with other information, in order to provide therapeutic interventional help to the users.

One or more patient devices (e.g., patient mobile computing devices, non-mobile computing devices, etc.), care provider devices, remote servers, and/or other suitable computing systems can be communicably connected (e.g., wired, wirelessly) through any suitable communication networks. For example, a remote server can be configured to receive a log of use dataset and/or a supplemental dataset (e.g., mobility behavior dataset) collected at a patient mobile computing device (e.g., a smartphone of the patient); to receive a survey dataset at a different patient mobile computing device (e.g., a tablet of the patient); to receive a care provider dataset collected at a care provider device (e.g., a care provider mobile computing device); to leverage such data in selecting a therapeutic intervention; generating a dynamic care plan; and/or dynamically modifying a dynamic care plan; and/or to promote a therapeutic intervention and/or a dynamic care plan at any one of the patient mobile computing devices and/or care provider mobile computing devices. In another example, a non-generalized mobile computing device (e.g., internet-enabled smartphone including a mobility-related sensor) can be configured to collect a log of use dataset (describing digital communication behaviors unique to computer network technology) and/or a mobility behavior dataset, and to receive additional supplementary datasets (e.g., cardiovascular device data, smart appliances data, smart light bulb data, etc.); and to leverage such data in selecting and/or promoting a therapeutic intervention, generating and/or dynamically modify a dynamic care plan, and/or implement other portions fo the method 100. However, the system 200 can include any suitable configuration of non-generalized computing systems connected in any combination to one or more communication networks.

While some variations of machine learning techniques are described above, portions of the method 100 (e.g., selecting a therapeutic intervention, generating a dynamic care plan, dynamically modifying a dynamic care plan, etc.) and/or system components implementing portions fo the method 100 can implement learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the machine learning algorithm can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4-5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, boostrapped aggregation, AdaBoost, stacked generalization, gradient boosting machine method, random forest method, etc.), and any suitable form of machine learning algorithm.

The processing system 205 and data handling by the modules of the processing system 205 are preferably adherent to health-related privacy laws (e.g., HIPAA), and are preferably configured to privatize and/or anonymize individual data according to encryption protocols. In an example, when an individual installs and/or authorizes collection and transmission of personal communication data by the system 200 through the native data collection application, the native application can prompt the individual to create a profile or account. In the example, the account can be stored locally on the individual's mobile computing device 209, and/or remotely. Furthermore, data processed or produced by modules of the system 200 can be configured to facilitate storage of data locally (e.g., on the patent's mobile computing device, in a remote database), or in any other suitable manner. For example, private health state-related data can be stored temporarily on the user's mobile computing device in a locked and encrypted file folder on integrated or removable memory. In this example, the user's data can be encrypted and uploaded to the remote database once a secure Internet connection is established. However, individual data can be stored on any other local device or remote data in any other suitable way and transmitted between the two over any other connection via any other suitable communication and/or encryption protocol. As such, the modules of the system 200 can be configured to perform embodiments, variations, and examples of the method 100 described above, in a manner that adheres to privacy-related health regulations.

The method 100 and/or system 200 of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of an individual computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

The FIGURES illustrate the architecture, functionality and operation of possible implementations of systems, methods and computer program products according to preferred embodiments, example configurations, and variations thereof. In this regard, each block in the flowchart or block diagrams can represent a module, segment, step, or portion of code, which includes one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The embodiments include every combination and permutation of the various system components and the various method processes, including any variations, examples, and specific examples.

As a person skilled in the art will recognize from the previous detailed description and from the figures and claims, modifications and changes can be made to the embodiments of the invention without departing from the scope of this invention as defined in the following claims.

We claim:

1. A system for automatically promoting an outcome to a user, the system comprising:
a client application executable on a mobile computing device associated with the user, wherein the mobile computing device comprises a sensor, wherein the mobile computing device collects a set of datasets, the set of datasets comprising:
a first passive dataset associated with digital patterns of the user at the mobile computing device;
a second passive dataset collected from the sensor;
a first active dataset collected from the client application, wherein the first active dataset comprises a set of inputs from the user;
a set of trained models; and
a computing system comprising the set of trained models and in communication with the mobile computing device, wherein the computing system:
generates a dynamic plan for the user with the set of trained models and based on the set of datasets, wherein the dynamic plan comprises a first output to the user, wherein the first output is conveyable at the mobile computing device;
automatically promotes the first output to the user at the mobile computing device;
receives a second set of inputs from the user in response to the first output;
with the set of trained models, automatically generates a second dynamic plan for the user based on the second set of inputs; and
automatically promotes a second output to the user at the mobile computing device in accordance with the second dynamic plan.

2. The system of claim 1, wherein the first output to the user is provided at a first time period, wherein the first time period is determined based on a set of time periods associated with the set of datasets.

3. The system of claim 2, wherein the first time period is determined with the set of trained models.

4. The system of claim 1, wherein the set of digital patterns comprises at least one of: a frequency of messages sent by the user from the mobile computing device, a length of messages sent by the user from the mobile computing device, and a ratio of inbound messages received at the mobile computing device versus outbound messages sent from the mobile computing device.

5. The system of claim 1, wherein the computing system further determines a set of feature vectors based on the first and second passive datasets and the first active dataset, wherein the set of feature vectors is determined based on a factor analysis approach with a linking analysis

6. The system of claim 1, wherein the computing sys tem comprises a remote computing subsystem in communication with the mobile computing device.

7. A method for automatically promoting an outcome to a user, the method comprising:
receiving a first passive dataset from a mobile computing device of the user, wherein the first passive dataset comprises a log of use dataset associated with digital behavior of the user at the mobile computing device, wherein the log of use dataset corresponds to a first time period;
receiving a second passive dataset from the mobile computing device, wherein the second passive dataset is collected from a sensor of the mobile computing device, wherein the second passive dataset corresponds to a second time period;
receiving a first active dataset from a client application executable on the mobile computing device, wherein the first active dataset comprises a set of entries from the user, wherein the first active dataset corresponds to a third time period;
with a set of trained models, generating a dynamic plan for the user, comprising determining a first output for the user based on processing the first passive dataset, the second passive dataset, and the first active dataset;
automatically promoting the first output to the user at the mobile computing device at a fourth time period, wherein the fourth time period is determined based on the first, second, and third time periods;
receiving a set of inputs from the user in response to the first output;
with the set of trained models, automatically generating a second dynamic plan for the user based on the set of inputs; and
automatically promoting a second output to the user at the mobile computing device in accordance with the second dynamic plan.

8. The method of claim 7, wherein the trained model comprises a machine learning model.

9. The method of claim 7, further comprising determining a set of feature vectors based on the first and second passive datasets and the first active dataset, wherein the set of feature vectors is determined based on a factor analysis approach with a linking analysis

10. The method of claim 7, further comprising receiving a second active dataset from a second user, wherein the dynamic plan, for the user is further determined based on the second active dataset.

11. The method of claim 10, wherein the first user is assigned to the second user as a part of a program.

12. The method of claim 7, wherein the fourth time period is further determined with the set of trained models.

13. The method of claim 12, wherein the fourth time period is further determined based on historical information associated with the user.

14. The method of claim 13, wherein the historical information comprises a set of interactions of the user with the client application.

15. The method of claim 7, wherein the log of use dataset comprises a number of inbound messages received at the mobile computing device and a number of outbound messages sent from the mobile computing device.

16. The method of claim 7, wherein the set of entries is collected from a set of digital surveys.

17. The method of claim 16, wherein the dynamic care plan comprises a second set of digital surveys.

18. A method for automatically promoting an outcome to a user, the method comprising:
receiving a set of datasets from a mobile computing device of the user, wherein the set of datasets comprises:
a first passive dataset associated with digital behavior of the user at the mobile computing device;
a second passive dataset from a sensor of the mobile computing device;
an active dataset from a client application executable on the mobile computing device, wherein the first active dataset comprises a set of entries from the user;
with a set of trained models, generating a dynamic plan for the user, comprising determining a first output for the user based on processing the set of datasets;
automatically promoting the first output to the user at the mobile computing device at a particular time period, wherein the particular time period is determined based on a set of time periods associated with the set of datasets;
receiving a set of inputs from the user in response to the first output;
with the set of trained models, automatically generating a second dynamic plan for the user based on the set of inputs; and
automatically promoting a second output to the user at the mobile computing device in accordance with the second dynamic plan.

19. The method of claim 18, further comprising receiving a second active dataset from a second user, wherein the dynamic plan for the user is further determined based on the second active dataset.

20. The method of claim 19, wherein the first user is assigned to the second user as a part of a program.
